SEQUENCE LISTING (182 sequences)

SEQ ID NO: 1 - protein, 906 aa, Artificial Sequence
  Synthetic Peptide
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 2 - protein, 121 aa, Artificial Sequence
  Synthetic Peptide
TSMKPINEDLDEGIMVVYKRNIAGSGCQLTFWEASERTIRSEAEDSYHFSSAKMTATFLSKKQEVNMSDSALDCVRDEAINKLQQIFNTSYNQTYEKYGNVSVFETSGGLVVFWQGIKQKS

SEQ ID NO: 3 - protein, 452 aa, Artificial Sequence
  Synthetic Peptide
QVQLQESGPGLVKPLETLSLTCTVSGGSISSGSFYWTWIRQHPGKGLEWIGYIHSSGTTYYNPSLKSRLNISRDTSKNQFTLKLSSVTAADTAVYYCAKEIIQWPRRWFDPWGQGTLVTVSSASTKGPSVRPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 455 aa, Artificial Sequence
  Synthetic Peptide
EVQLLESGGGLIQPGGSLRLSCVASGFTFTRHAINWVRQAPGKGLEWVSAISGSGSSTYYADSVKGRFTISRDNAKNTLYLQMNRLRVEDTAVYYCAKDKDYGMVAATPDAFDIWGQGTMVTVSSASTKGPSVRPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 454 aa, Artificial Sequence
  Synthetic Peptide
QVQLQESGPGLVKPLETLSLTCTVSGGSIRGGDYWWTWVRQPPGKGLEWLGYIYRTGSTYYNPSLKSRLTISLDTSKNHFSLKLASATAADTAVYYCARDPGDDSGGNSGLDYWGQGVLVSVSSASTKGPSVRPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 453 aa, Artificial Sequence
  Synthetic Peptide
QVQLVESGGGLVKPGGSLRLSCTASGLTFSDYYLSWIRQAPGKGLEWISYISGFNTYTNYADSVRGRFTISRDNAKKSLYLQMDSLRVEDTAVYYCTGARRGESGAYGSSDYWGLGTLVTVSSASTKGPSVRPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 452 aa, Artificial Sequence
  Synthetic Peptide
EMQLLESGGGLVQPGGSLRVSCAASGFTFSSHAMSWVRQAPGKGLEWVSSISRSGDNTFYADSVKGRFTISRDNIKSTVYLQMNSLRAEDTAVYFCASRYTEGDSVWYFDVWGRGTLVTVSSASTKGPSVRPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial Sequence
  Synthetic Peptide
LFVLTQPPSASGTAGQRVTISCSASNSNIGTNTVTWYQHLPGAAPRLLIYNNDQRPSGVPDRFSGSRSGTSASLAISGLQSEDETVYYCSAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 9 - protein, 216 aa, Artificial Sequence
  Synthetic Peptide
LFVLTQPPSASGTPGQRVTISCSASSSSIGTNTVTWYQHLPGAAPKLLIYNNDQRPSGIPDRFSGSKSGTSASLAITGLQSEDETVYYCAAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 10 - protein, 216 aa, Artificial Sequence
  Synthetic Peptide
QSMLTQPPSVSAAPGQKVIISCSGSSSNIGNNYVNWYQQLPGTAPKLLIYDNDKRPSGIPDRFSGSKSGTSATLGITGLQTGDEADYYCGTWDSTLTAGVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 11 - protein, 216 aa, Artificial Sequence
  Synthetic Peptide
QSVLTQPPSVSAAPGQKVIISCSGSGSNIGSHYVNWYQQLPGAAPKLLIYDNDKRPSGIPDRFSGSKSGTSATLAITGLQTGDEADYHCATWDGTLTAGVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 12 - protein, 216 aa, Artificial Sequence
  Synthetic Peptide
QSMLTQPPSVSAAPGQKVIISCSGSSSNIGNNFVNWYQKFPGTAPKLLIYDNDKRPSGIPDRFSGSKSGTSATLGITGLQTGDEAEYYCGTWDSSLTAGVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 13 - protein, 216 aa, Artificial Sequence
  Synthetic Peptide
QSVLTQPPSASGTPGQRVTISCSGSSSNIGSNTVTWYQQLPGTAPKLLISRNNQRPSGVPDRFSGSKSGTSASLAISGLQSEDEADYYCSSWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 14 - protein, 216 aa, Artificial Sequence
  Synthetic Peptide
QSVLTQPPSASGTPGQRVTISCSGSSSNIGSNTVNWYQQVPGTAPRLLIYSHNQRPSGVPDRFSGSKSGTSASLAISGLQSEDEADYYCASWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 15 - protein, 217 aa, Artificial Sequence
  Synthetic Peptide
QSVLTQPPSVSAAPGQKVTISCSGSRSNIGKNYVYWYQQLPGTAPKLLIYDNNKRPSGIPDRFSGSKSGTSATLAITGLQTGDEADYYCGTWDSSLKTGIFFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 16 - protein, 217 aa, Artificial Sequence
  Synthetic Peptide
QSVLTQPPSVSAAPGQKVTISCSGSRSNIAKNYVYWYQQLPGTAPKLLIYDTNKRPSGIPDRFSGSKSGTSATLAITGLQTGDEADYYCGTWDSSLKTAIFFGGGTTVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 17 - protein, 216 aa, Artificial Sequence
  Synthetic Peptide
QSVLTQPPSASGTPGQRVTISCSGSSSNIGSNYIDWYQQLPGTGPQLLTYRNNQRPSGVPDRFSGSKSGTSASLAISGLRSEDEADYYCASWDDSLSSPVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 18 - protein, 214 aa, Artificial Sequence
  Synthetic Peptide
AIRMTQSPSSFSASTGDRVTITCRASQGISNYLAWYQQKPGKAPKLLIYAASTLQSGVPSRFSGSGSGTDFTLTISCLQSEDFATYYCQQYYSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, Artificial Sequence; identical to SEQ ID NO: 18
  Synthetic Peptide

SEQ ID NO: 20 - protein, 214 aa, Artificial Sequence
  Synthetic Peptide
EVVLTQSPATLSVSAGDRATLSCRASQSVSRDLAWYQQKPGQAPRLLIYGASTRATDIPVRFSGSGSGTEFSLTISSLQSEDFAVYYCHQYKHWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 366 bp, Artificial Sequence
  Synthetic Polynucleotide
caggtgcagctgcaggagtcgggcccaggactggtgaagcctttagagaccctgtccctcacctgcactgtctctggtggctccatcagcagtggtagtttctactggacctggatccgccaacacccagggaagggcctggagtggattgggtacatccattccagtggcaccacctactacaacccgtccctcaagagtcgacttaacatatcaagagacacgtctaagaaccagttcaccctgaaactgagctcggtgactgccgcggacacggccgtctattactgtgcgaaagagattatacaatggccccggcggtggttcgacccctggggccagggaaccctggtcaccgtctcctca

SEQ ID NO: 22 - protein, 122 aa, Artificial Sequence
  Synthetic Peptide
QVQLQESGPGLVKPLETLSLTCTVSGGSISSGSFYWTWIRQHPGKGLEWIGYIHSSGTTYYNPSLKSRLNISRDTSKNQFTLKLSSVTAADTAVYYCAKEIIQWPRRWFDPWGQGTLVTVSS

SEQ ID NO: 23 - protein, 30 aa, Artificial Sequence
  Synthetic Peptide
QVQLQESGPGLVKPLETLSLTCTVSGGSIS

SEQ ID NO: 24 - protein, 7 aa, Artificial Sequence
  Synthetic Peptide
SGSFYWT

SEQ ID NO: 25 - protein, 14 aa, Artificial Sequence
  Synthetic Peptide
WIRQHPGKGLEWIG

SEQ ID NO: 26 - protein, 16 aa, Artificial Sequence
  Synthetic Peptide
YIHSSGTTYYNPSLKS

SEQ ID NO: 27 - protein, 32 aa, Artificial Sequence
  Synthetic Peptide
RLNISRDTSKNQFTLKLSSVTAADTAVYYCAK

SEQ ID NO: 28 - protein, 12 aa, Artificial Sequence
  Synthetic Peptide
EIIQWPRRWFDP

SEQ ID NO: 29 - protein, 11 aa, Artificial Sequence
  Synthetic Peptide
WGQGTLVTVSS

SEQ ID NO: 30 - DNA, 375 bp, Artificial Sequence
  Synthetic Polynucleotide
gaggtgcagctgttggagtctgggggaggcttgatacagcctggggggtccctgagactctcctgtgtagcctctggattcacctttactagacatgccataaattgggtccgccaggctccagggaaggggctggagtgggtctcagctattagtggtagtggtagtagcacatattacgcagactccgtgaagggccgattcaccatctccagagacaatgccaagaatacgctgtatctgcaaatgaacagactgcgagtcgaggacacggccgtgtattactgtgcgaaagataaggattacgggatggtagccgctacaccagatgcttttgatatctggggccaagggacaatggtcaccgtctcttca

SEQ ID NO: 31 - protein, 125 aa, Artificial Sequence
  Synthetic Peptide
EVQLLESGGGLIQPGGSLRLSCVASGFTFTRHAINWVRQAPGKGLEWVSAISGSGSSTYYADSVKGRFTISRDNAKNTLYLQMNRLRVEDTAVYYCAKDKDYGMVAATPDAFDIWGQGTMVTVSS

SEQ ID NO: 32 - protein, 30 aa, Artificial Sequence
  Synthetic Peptide
EVQLLESGGGLIQPGGSLRLSCVASGFTFT

SEQ ID NO: 33 - protein, 5 aa, Artificial Sequence
  Synthetic Peptide
RHAIN

SEQ ID NO: 34 - protein, 14 aa, Artificial Sequence
  Synthetic Peptide
WVRQAPGKGLEWVS

SEQ ID NO: 35 - protein, 17 aa, Artificial Sequence
  Synthetic Peptide
AISGSGSSTYYADSVKG

SEQ ID NO: 36 - protein, 32 aa, Artificial Sequence
  Synthetic Peptide
RFTISRDNAKNTLYLQMNRLRVEDTAVYYCAK

SEQ ID NO: 37 - protein, 16 aa, Artificial Sequence
  Synthetic Peptide
DKDYGMVAATPDAFDI

SEQ ID NO: 38 - protein, 11 aa, Artificial Sequence
  Synthetic Peptide
WGQGTMVTVSS

SEQ ID NO: 39 - DNA, 372 bp, Artificial Sequence
  Synthetic Polynucleotide
caggtgcagttgcaggagtcgggcccaggactggtgaagcctttagagaccctgtccctcacctgcactgtgtctggtggctccatcagaggtggtgattactggtggacttgggtccgccagcccccaggtaagggcctggagtggcttggctacatatatcgcactgggagcacctactacaatccgtccctcaagagtcgacttacgatctcattggacacgtccaagaaccacttctccctgaagctggcctcggctactgccgcagacacggccgtctattactgtgccagagacccaggtgatgactccggtggaaactcgggcttggactactggggccagggagtcctcgtctccgtctcctca

SEQ ID NO: 40 - protein, 124 aa, Artificial Sequence
  Synthetic Peptide
QVQLQESGPGLVKPLETLSLTCTVSGGSIRGGDYWWTWVRQPPGKGLEWLGYIYRTGSTYYNPSLKSRLTISLDTSKNHFSLKLASATAADTAVYYCARDPGDDSGGNSGLDYWGQGVLVSVSS

SEQ ID NO: 41 - protein, 30 aa, Artificial Sequence
  Synthetic Peptide
QVQLQESGPGLVKPLETLSLTCTVSGGSIR

SEQ ID NO: 42 - protein, 7 aa, Artificial Sequence
  Synthetic Peptide
GGDYWWT

SEQ ID NO: 43 - protein, 14 aa, Artificial Sequence
  Synthetic Peptide
WVRQPPGKGLEWLG

SEQ ID NO: 44 - protein, 16 aa, Artificial Sequence
  Synthetic Peptide
YIYRTGSTYYNPSLKS

SEQ ID NO: 45 - protein, 32 aa, Artificial Sequence
  Synthetic Peptide
RLTISLDTSKNHFSLKLASATAADTAVYYCAR

SEQ ID NO: 46 - protein, 14 aa, Artificial Sequence
  Synthetic Peptide
DPGDDSGGNSGLDY

SEQ ID NO: 47 - protein, 11 aa, Artificial Sequence
  Synthetic Peptide
WGQGVLVSVSS

SEQ ID NO: 48 - DNA, 369 bp, Artificial Sequence
  Synthetic Polynucleotide
caggtgcagctggtggagtctgggggaggcttggtcaagcctggagggtccctgagactctcctgtacagcctctggattgaccttcagtgactactacctgagctggatccgccaggctccagggaaggggctggagtggatttcatacattagtggtttcaatacttacacaaactatgcagactctgtgaggggccgattcaccatctccagagacaacgccaagaaatcactgtatctgcaaatggacagcctgagagtcgaggacacggctgtctattattgtacgggggcccgcaggggcgagagtggggcctacgggtcgagtgactactggggcctgggaaccctggtcaccgtctcctca

SEQ ID NO: 49 - protein, 123 aa, Artificial Sequence
  Synthetic Peptide
QVQLVESGGGLVKPGGSLRLSCTASGLTFSDYYLSWIRQAPGKGLEWISYISGFNTYTNYADSVRGRFTISRDNAKKSLYLQMDSLRVEDTAVYYCTGARRGESGAYGSSDYWGLGTLVTVSS

SEQ ID NO: 50 - protein, 30 aa, Artificial Sequence
  Synthetic Peptide
QVQLVESGGGLVKPGGSLRLSCTASGLTFS

SEQ ID NO: 51 - protein, 5 aa, Artificial Sequence
  Synthetic Peptide
DYYLS

SEQ ID NO: 52 - protein, 14 aa, Artificial Sequence
  Synthetic Peptide
WIRQAPGKGLEWIS

SEQ ID NO: 53 - protein, 17 aa, Artificial Sequence
  Synthetic Peptide
YISGFNTYTNYADSVRG

SEQ ID NO: 54 - protein, 32 aa, Artificial Sequence
  Synthetic Peptide
RFTISRDNAKKSLYLQMDSLRVEDTAVYYCTG

SEQ ID NO: 55 - protein, 14 aa, Artificial Sequence
  Synthetic Peptide
ARRGESGAYGSSDY

SEQ ID NO: 56 - protein, 11 aa, Artificial Sequence
  Synthetic Peptide
WGLGTLVTVSS

SEQ ID NO: 57 - DNA, 366 bp, Artificial Sequence
  Synthetic Polynucleotide
gagatgcagctgttggagtctgggggaggcttggtacagcctggggggtccctgagagtctcctgtgcagcctctggattcacctttagcagtcatgccatgagttgggtccgccaggctccagggaaggggctggagtgggtctcaagtataagtcgaagtggtgataacacattctacgcagactccgtgaagggccgcttcaccatctccagagacaacattaagagtacagtgtatctgcaaatgaacagcctgagagccgaggacacggccgtatatttctgtgcgagtcgatatacggaaggtgactccgtctggtacttcgatgtctggggccggggcaccctggtcactgtctcctca

SEQ ID NO: 58 - protein, 122 aa, Artificial Sequence
  Synthetic Peptide
EMQLLESGGGLVQPGGSLRVSCAASGFTFSSHAMSWVRQAPGKGLEWVSSISRSGDNTFYADSVKGRFTISRDNIKSTVYLQMNSLRAEDTAVYFCASRYTEGDSVWYFDVWGRGTLVTVSS

SEQ ID NO: 59 - protein, 30 aa, Artificial Sequence
  Synthetic Peptide
EMQLLESGGGLVQPGGSLRVSCAASGFTFS

SEQ ID NO: 60 - protein, 5 aa, Artificial Sequence
  Synthetic Peptide
SHAMS

SEQ ID NO: 61 - protein, 14 aa, Artificial Sequence
  Synthetic Peptide
WVRQAPGKGLEWVS

SEQ ID NO: 62 - protein, 17 aa, Artificial Sequence
  Synthetic Peptide
SISRSGDNTFYADSVKG

SEQ ID NO: 63 - protein, 32 aa, Artificial Sequence
  Synthetic Peptide
RFTISRDNIKSTVYLQMNSLRAEDTAVYFCAS

SEQ ID NO: 64 - protein, 13 aa, Artificial Sequence
  Synthetic Peptide
RYTEGDSVWYFDV

SEQ ID NO: 65 - protein, 11 aa, Artificial Sequence
  Synthetic Peptide
WGRGTLVTVSS

SEQ ID NO: 66 - DNA, 339 bp, Artificial Sequence
  Synthetic Polynucleotide
ctgtttgtgttgactcagccgccctcagcgtcagggaccgccgggcagagggtcaccatctcttgttctgcaagcaactccaacatcggaactaatactgtaacctggtaccagcatctcccaggagcggcccccagactcctcatctataataatgatcagaggccctcaggggtccctgaccgattctctggctccaggtctggcacctcagcctccctggccatcagtgggctccagtctgaggatgagactgtttattactgttcagcatgggatgacagcctgaatggcgtggtgttcggcggagggaccaaactgaccgtcctaggtcagccc

SEQ ID NO: 67 - protein, 110 aa, Artificial Sequence
  Synthetic Peptide
LFVLTQPPSASGTAGQRVTISCSASNSNIGTNTVTWYQHLPGAAPRLLIYNNDQRPSGVPDRFSGSRSGTSASLAISGLQSEDETVYYCSAWDDSLNGVVFGGGTKLTVL

SEQ ID NO: 68 - protein, 22 aa, Artificial Sequence
  Synthetic Peptide
LFVLTQPPSASGTAGQRVTISC

SEQ ID NO: 69 - protein, 13 aa, Artificial Sequence
  Synthetic Peptide
SASNSNIGTNTVT

SEQ ID NO: 70 - protein, 15 aa, Artificial Sequence
  Synthetic Peptide
WYQHLPGAAPRLLIY

SEQ ID NO: 71 - protein, 7 aa, Artificial Sequence
  Synthetic Peptide
NNDQRPS

SEQ ID NO: 72 - protein, 32 aa, Artificial Sequence
  Synthetic Peptide
GVPDRFSGSRSGTSASLAISGLQSEDETVYYC

SEQ ID NO: 73 - protein, 10 aa, Artificial Sequence
  Synthetic Peptide
AWDDSLNGVV

SEQ ID NO: 74 - protein, 10 aa, Artificial Sequence
  Synthetic Peptide
FGGGTKLTVL

SEQ ID NO: 75 - DNA, 339 bp, Artificial Sequence
  Synthetic Polynucleotide
ctctttgtgttgactcagccaccctcagcgtcagggacccccgggcagagggtcaccatctcttgttctgcaagcagctccagcatcggaactaacaccgtaacctggtaccagcatctcccaggagcggcccccaaactcctaatctataataatgatcagcggccctcagggatccctgaccgattttctggctccaagtctggcacctcagcctccctggccatcactgggctccagtctgaggatgagactgtttattactgtgcagcatgggatgacagtctgaatggcgtggttttcggcggagggaccaagctgaccgtcctaggtcagccc

SEQ ID NO: 76 - protein, 110 aa, Artificial Sequence
  Synthetic Peptide
LFVLTQPPSASGTPGQRVTISCSASSSSIGTNTVTWYQHLPGAAPKLLIYNNDQRPSGIPDRFSGSKSGTSASLAITGLQSEDETVYYCAAWDDSLNGVVFGGGTKLTVL

SEQ ID NO: 77 - protein, 22 aa, Artificial Sequence
  Synthetic Peptide
LFVLTQPPSASGTPGQRVTISC

SEQ ID NO: 78 - protein, 13 aa, Artificial Sequence
  Synthetic Peptide
SASSSSIGTNTVT

SEQ ID NO: 79 - protein, 15 aa, Artificial Sequence
  Synthetic Peptide
WYQHLPGAAPKLLIY

SEQ ID NO: 80 - protein, 7 aa, Artificial Sequence
  Synthetic Peptide
NNDQRPS

SEQ ID NO: 81 - protein, 32 aa, Artificial Sequence
  Synthetic Peptide
GIPDRFSGSKSGTSASLAITGLQSEDETVYYC

SEQ ID NO: 82 - protein, 11 aa, Artificial Sequence
  Synthetic Peptide
AAWDDSLNGVV

SEQ ID NO: 83 - protein, 10 aa, Artificial Sequence
  Synthetic Peptide
FGGGTKLTVL

SEQ ID NO: 84 - DNA, 339 bp, Artificial Sequence
  Synthetic Polynucleotide
cagtctatgttgacgcagccgccctcagtgtctgcggccccaggacagaaggtcatcatctcctgctctggaagcagctccaacattgggaataactatgtgaactggtaccagcagctcccaggaacagcccccaaactcctcatttatgacaatgataagcgaccctcagggattcctgaccgattctctggctccaagtctggcacgtcggccaccctgggcatcaccggactccagactggggacgaggccgattattactgcggaacatgggatagcaccctgactgcgggggtgttcggcggagggaccaagttgaccgtcctgggtcagccc

SEQ ID NO: 85 - protein, 110 aa, Artificial Sequence
  Synthetic Peptide
QSMLTQPPSVSAAPGQKVIISCSGSSSNIGNNYVNWYQQLPGTAPKLLIYDNDKRPSGIPDRFSGSKSGTSATLGITGLQTGDEADYYCGTWDSTLTAGVFGGGTKLTVL

SEQ ID NO: 86 - protein, 22 aa, Artificial Sequence
  Synthetic Peptide
QSMLTQPPSVSAAPGQKVIISC

SEQ ID NO: 87 - protein, 13 aa, Artificial Sequence
  Synthetic Peptide
SGSSSNIGNNYVN

SEQ ID NO: 88 - protein, 15 aa, Artificial Sequence
  Synthetic Peptide
WYQQLPGTAPKLLIY

SEQ ID NO: 89 - protein, 7 aa, Artificial Sequence
  Synthetic Peptide
DNDKRPS

SEQ ID NO: 90 - protein, 32 aa, Artificial Sequence
  Synthetic Peptide
GIPDRFSGSKSGTSATLGITGLQTGDEADYYC

SEQ ID NO: 91 - protein, 11 aa, Artificial Sequence
  Synthetic Peptide
GTWDSTLTAGV

SEQ ID NO: 92 - protein, 10 aa, Artificial Sequence
  Synthetic Peptide
FGGGTKLTVL

SEQ ID NO: 93 - DNA, 339 bp, Artificial Sequence
  Synthetic Polynucleotide
cagtctgtcttgacacagccgccctcagtgtctgcggcccctggacagaaggtcatcatttcctgctctggcagcggctccaacattggcagtcattatgtaaattggtatcagcagctcccaggagcagcccccaaactcctcatttatgacaatgataagcgaccctcagggattcctgaccgattctctggctccaagtctggcacgtcagccaccctggccatcaccggactccagactggggacgaggccgactatcactgcgcaacatgggatggcaccctcactgcgggggtgttcggcggagggaccaagctgaccgtcctaggtcagccc

SEQ ID NO: 94 - protein, 110 aa, Artificial Sequence
  Synthetic Peptide
QSVLTQPPSVSAAPGQKVIISCSGSGSNIGSHYVNWYQQLPGAAPKLLIYDNDKRPSGIPDRFSGSKSGTSATLAITGLQTGDEADYHCATWDGTLTAGVFGGGTKLTVL

SEQ ID NO: 95 - protein, 22 aa, Artificial Sequence
  Synthetic Peptide
QSVLTQPPSVSAAPGQKVIISC

SEQ ID NO: 96 - protein, 13 aa, Artificial Sequence
  Synthetic Peptide
SGSGSNIGSHYVN

SEQ ID NO: 97 - protein, 15 aa, Artificial Sequence
  Synthetic Peptide
WYQQLPGAAPKLLIY

SEQ ID NO: 98 - protein, 7 aa, Artificial Sequence
  Synthetic Peptide
DNDKRPS

SEQ ID NO: 99 - protein, 32 aa, Artificial Sequence
  Synthetic Peptide
GIPDRFSGSKSGTSATLAITGLQTGDEADYHC

SEQ ID NO: 100 - protein, 11 aa, Artificial Sequence
  Synthetic Peptide
ATWDGTLTAGV

SEQ ID NO: 101 - protein, 10 aa, Artificial Sequence
  Synthetic Peptide
FGGGTKLTVL

SEQ ID NO: 102 - DNA, 339 bp, Artificial Sequence
  Synthetic Polynucleotide
cagtctatgttgacgcagccgccctcagtgtctgcggccccaggacagaaggtcatcatctcctgctctggaagcagctccaacattggaaataactttgtaaactggtaccagaagttcccaggaacagcccccaaactcctcatttatgacaatgataagcgaccctcagggattcctgaccgattctctggctccaagtccggcacgtcagccaccctgggcatcaccggactccagactggggacgaggccgaatattattgcggaacatgggatagcagcctgactgcgggggtgttcggcggagggaccacgctgaccgtcctgggtcagccc

SEQ ID NO: 103 - protein, 110 aa, Artificial Sequence
  Synthetic Peptide
QSMLTQPPSVSAAPGQKVIISCSGSSSNIGNNFVNWYQKFPGTAPKLLIYDNDKRPSGIPDRFSGSKSGTSATLGITGLQTGDEAEYYCGTWDSSLTAGVFGGGTTLTVL

SEQ ID NO: 104 - protein, 22 aa, Artificial Sequence
  Synthetic Peptide
QSMLTQPPSVSAAPGQKVIISC

SEQ ID NO: 105 - protein, 13 aa, Artificial Sequence
  Synthetic Peptide
SGSSSNIGNNFVN

SEQ ID NO: 106 - protein, 15 aa, Artificial Sequence
  Synthetic Peptide
WYQKFPGTAPKLLIY

SEQ ID NO: 107 - protein, 7 aa, Artificial Sequence
  Synthetic Peptide
DNDKRPS

SEQ ID NO: 108 - protein, 32 aa, Artificial Sequence
  Synthetic Peptide
GIPDRFSGSKSGTSATLGITGLQTGDEAEYYC

SEQ ID NO: 109 - protein, 11 aa, Artificial Sequence
  Synthetic Peptide
GTWDSSLTAGV

SEQ ID NO: 110 - protein, 10 aa, Artificial Sequence
  Synthetic Peptide
FGGGTTLTVL

SEQ ID NO: 111 - DNA, 339 bp, Artificial Sequence
  Synthetic Polynucleotide
cagtctgtgctgactcagccaccctcagcgtctgggacccccgggcagagggtcaccatctcttgttctggaagcagctccaacatcggaagtaatactgtaacctggtaccagcagctcccaggaacggcccccaaactcctcatctctcgtaataatcagcggccctcaggggtccctgaccgattctctggctccaagtctggcacctcagcctccctggccatcagtgggctccagtctgaggatgaggctgattattattgttcatcatgggatgacagcctgaatggtgtggtgttcggcggagggaccaagctgaccgtcctaggtcagccc

SEQ ID NO: 112 - protein, 110 aa, Artificial Sequence
  Synthetic Peptide
QSVLTQPPSASGTPGQRVTISCSGSSSNIGSNTVTWYQQLPGTAPKLLISRNNQRPSGVPDRFSGSKSGTSASLAISGLQSEDEADYYCSSWDDSLNGVVFGGGTKLTVL

SEQ ID NO: 113 - protein, 22 aa, Artificial Sequence
  Synthetic Peptide
QSVLTQPPSASGTPGQRVTISC

SEQ ID NO: 114 - protein, 13 aa, Artificial Sequence
  Synthetic Peptide
SGSSSNIGSNTVT

SEQ ID NO: 115 - protein, 15 aa, Artificial Sequence
  Synthetic Peptide
WYQQLPGTAPKLLIS

SEQ ID NO: 116 - protein, 7 aa, Artificial Sequence
  Synthetic Peptide
RNNQRPS

SEQ ID NO: 117 - protein, 32 aa, Artificial Sequence
  Synthetic Peptide
GVPDRFSGSKSGTSASLAISGLQSEDEADYYC

SEQ ID NO: 118 - protein, 11 aa, Artificial Sequence
  Synthetic Peptide
SSWDDSLNGVV

SEQ ID NO: 119 - protein, 10 aa, Artificial Sequence
  Synthetic Peptide
FGGGTKLTVL

SEQ ID NO: 120 - DNA, 339 bp, Artificial Sequence
  Synthetic Polynucleotide
cagtctgtgctgactcagccaccctcagcgtctgggacccccgggcagagggtcaccatctcttgttctgggagcagctccaacatcggaagtaatactgtaaactggtaccagcaggtcccaggaacggcccccagactcctcatctatagtcataatcagcggccctcaggggtccctgaccgattctctggctccaagtctggcacctcagcctccctggccatcagtggcctccagtctgaggatgaggctgattattactgtgcatcatgggatgacagcctgaatggtgtggtattcggcggagggaccaagctgaccgtcctaggtcagccc

SEQ ID NO: 121 - protein, 110 aa, Artificial Sequence
  Synthetic Peptide
QSVLTQPPSASGTPGQRVTISCSGSSSNIGSNTVNWYQQVPGTAPRLLIYSHNQRPSGVPDRFSGSKSGTSASLAISGLQSEDEADYYCASWDDSLNGVVFGGGTKLTVL

SEQ ID NO: 122 - protein, 22 aa, Artificial Sequence
  Synthetic Peptide
QSVLTQPPSASGTPGQRVTISC

SEQ ID NO: 123 - protein, 13 aa, Artificial Sequence
  Synthetic Peptide
SGSSSNIGSNTVN

SEQ ID NO: 124 - protein, 15 aa, Artificial Sequence
  Synthetic Peptide
WYQQVPGTAPRLLIY

SEQ ID NO: 125 - protein, 7 aa, Artificial Sequence
  Synthetic Peptide
SHNQRPS

SEQ ID NO: 126 - protein, 32 aa, Artificial Sequence; identical to SEQ ID NO: 117
  Synthetic Peptide

SEQ ID NO: 127 - protein, 11 aa, Artificial Sequence
  Synthetic Peptide
ASWDDSLNGVV

SEQ ID NO: 128 - protein, 10 aa, Artificial Sequence
  Synthetic Peptide
FGGGTKLTVL

SEQ ID NO: 129 - DNA, 342 bp, Artificial Sequence
  Synthetic Polynucleotide
cagtctgtgttgacgcagccgccctcagtgtctgcggccccgggacagaaggtcaccatctcctgctctggaagcagatccaacattgggaagaattatgtatactggtaccagcaactcccaggaacagcccccaaactcctcatttatgacaataataagcgaccctcagggattcctgaccgattctctggctccaagtctggcacgtcggccaccctggccatcaccggcctccagactggggacgaggccgattattactgcggaacatgggatagcagcctgaaaactggcatttttttcggcggagggaccaagctgaccgtcctaggtcagccc

SEQ ID NO: 130 - protein, 111 aa, Artificial Sequence
  Synthetic Peptide
QSVLTQPPSVSAAPGQKVTISCSGSRSNIGKNYVYWYQQLPGTAPKLLIYDNNKRPSGIPDRFSGSKSGTSATLAITGLQTGDEADYYCGTWDSSLKTGIFFGGGTKLTVL

SEQ ID NO: 131 - protein, 22 aa, Artificial Sequence
  Synthetic Peptide
QSVLTQPPSVSAAPGQKVTISC

SEQ ID NO: 132 - protein, 13 aa, Artificial Sequence
  Synthetic Peptide
SGSRSNIGKNYVY

SEQ ID NO: 133 - protein, 15 aa, Artificial Sequence
  Synthetic Peptide
WYQQLPGTAPKLLIY

SEQ ID NO: 134 - protein, 7 aa, Artificial Sequence
  Synthetic Peptide
DNNKRPS

SEQ ID NO: 135 - protein, 32 aa, Artificial Sequence
  Synthetic Peptide
GIPDRFSGSKSGTSATLAITGLQTGDEADYYC

SEQ ID NO: 136 - protein, 12 aa, Artificial Sequence
  Synthetic Peptide
GTWDSSLKTGIF

SEQ ID NO: 137 - protein, 10 aa, Artificial Sequence
  Synthetic Peptide
FGGGTKLTVL

SEQ ID NO: 138 - DNA, 342 bp, Artificial Sequence
  Synthetic Polynucleotide
cagtctgtgttgacgcagccgccctcagtgtctgcggccccgggacagaaggtcaccatctcctgctctggaagcagatccaacattgcgaagaattatgtatattggtatcaacaactcccaggaacagcccccaaactcctcatttatgacactaataagcgaccctcagggattcctgacagattctctggctccaagtctggcacgtcggccaccctggccatcaccggcctccagactggggacgaggccgattattactgcggaacatgggatagcagcctgaaaactgccatttttttcggcggagggaccacggtgaccgtcctaggtcagccc

SEQ ID NO: 139 - protein, 111 aa, Artificial Sequence
  Synthetic Peptide
QSVLTQPPSVSAAPGQKVTISCSGSRSNIAKNYVYWYQQLPGTAPKLLIYDTNKRPSGIPDRFSGSKSGTSATLAITGLQTGDEADYYCGTWDSSLKTAIFFGGGTTVTVL

SEQ ID NO: 140 - protein, 22 aa, Artificial Sequence
  Synthetic Peptide
QSVLTQPPSVSAAPGQKVTISC

SEQ ID NO: 141 - protein, 13 aa, Artificial Sequence
  Synthetic Peptide
SGSRSNIAKNYVY

SEQ ID NO: 142 - protein, 15 aa, Artificial Sequence
  Synthetic Peptide
WYQQLPGTAPKLLIY

SEQ ID NO: 143 - protein, 7 aa, Artificial Sequence
  Synthetic Peptide
DTNKRPS

SEQ ID NO: 144 - protein, 32 aa, Artificial Sequence; identical to SEQ ID NO: 135
  Synthetic Peptide

SEQ ID NO: 145 - protein, 12 aa, Artificial Sequence
  Synthetic Peptide
GTWDSSLKTAIF

SEQ ID NO: 146 - protein, 10 aa, Artificial Sequence
  Synthetic Peptide
FGGGTTVTVL

SEQ ID NO: 147 - DNA, 339 bp, Artificial Sequence
  Synthetic Peptide
cagtctgtactgactcagccaccctcagcgtctgggacccccgggcagagggtcaccatctcttgttctggaagcagctccaacatcggaagtaattatatagactggtaccagcagctcccaggaactggcccccaactcctcacttataggaacaatcagcggccctcaggggtccctgaccgcttctctggctccaagtctggcacctcagcctccctggccatcagtgggctccggtccgaggatgaggctgattattactgtgcatcatgggatgacagtctgagtagtcccgtattcggcggagggaccaagctgaccgtcctaggtcagccc

SEQ ID NO: 148 - protein, 110 aa, Artificial Sequence
  Synthetic Peptide
QSVLTQPPSASGTPGQRVTISCSGSSSNIGSNYIDWYQQLPGTGPQLLTYRNNQRPSGVPDRFSGSKSGTSASLAISGLRSEDEADYYCASWDDSLSSPVFGGGTKLTVL

SEQ ID NO: 149 - protein, 22 aa, Artificial Sequence
  Synthetic Peptide
QSVLTQPPSASGTPGQRVTISC

SEQ ID NO: 150 - protein, 13 aa, Artificial Sequence
  Synthetic Peptide
SGSSSNIGSNYID

SEQ ID NO: 151 - protein, 15 aa, Artificial Sequence
  Synthetic Peptide
WYQQLPGTGPQLLTY

SEQ ID NO: 152 - protein, 7 aa, Artificial Sequence
  Synthetic Peptide
RNNQRPS

SEQ ID NO: 153 - protein, 32 aa, Artificial Sequence
  Synthetic Peptide
GVPDRFSGSKSGTSASLAISGLRSEDEADYYC

SEQ ID NO: 154 - protein, 11 aa, Artificial Sequence
  Synthetic Peptide
ASWDDSLSSPV

SEQ ID NO: 155 - protein, 10 aa, Artificial Sequence
  Synthetic Peptide
FGGGTKLTVL

SEQ ID NO: 156 - DNA, 324 bp, Artificial Sequence
  Synthetic Polynucleotide
gccatccggatgacccagtctccatcctcattctctgcatctacaggagacagagtcaccatcacttgtcgggcgagtcagggtattagcaattatttagcctggtatcagcaaaaaccagggaaagcccctaagctcctgatctatgctgcatccactttgcaaagtggggtcccatcaaggttcagcggcagtggatctgggacagatttcactctcaccatcagctgcctgcagtctgaagattttgcaacttattactgtcaacagtattatagttacccattcactttcggccctgggaccaaagtggatatcaaacga

SEQ ID NO: 157 - protein, 107 aa, Artificial Sequence
  Synthetic Peptide
AIRMTQSPSSFSASTGDRVTITCRASQGISNYLAWYQQKPGKAPKLLIYAASTLQSGVPSRFSGSGSGTDFTLTISCLQSEDFATYYCQQYYSYPFTFGPGTKVDIK

SEQ ID NO: 158 - protein, 23 aa, Artificial Sequence
  Synthetic Peptide
AIRMTQSPSSFSASTGDRVTITC

SEQ ID NO: 159 - protein, 11 aa, Artificial Sequence
  Synthetic Peptide
RASQGISNYLA

SEQ ID NO: 160 - protein, 15 aa, Artificial Sequence
  Synthetic Peptide
WYQQKPGKAPKLLIY

SEQ ID NO: 161 - protein, 7 aa, Artificial Sequence
  Synthetic Peptide
AASTLQS

SEQ ID NO: 162 - protein, 32 aa, Artificial Sequence
  Synthetic Peptide
GVPSRFSGSGSGTDFTLTISCLQSEDFATYYC

SEQ ID NO: 163 - protein, 9 aa, Artificial Sequence
  Synthetic Peptide
QQYYSYPFT

SEQ ID NO: 164 - protein, 10 aa, Artificial Sequence
  Synthetic Peptide
FGPGTKVDIK

SEQ ID NO: 165 - DNA, 324 bp, Artificial Sequence; identical to SEQ ID NO: 156
  Synthetic Polynucleotide

SEQ ID NO: 166 - protein, 107 aa, Artificial Sequence; identical to SEQ ID NO: 157
  Synthetic Peptide

SEQ ID NO: 167 - protein, 23 aa, Artificial Sequence
  Synthetic Peptide
AIRMTQSPSSFSASTGDRVTITC

SEQ ID NO: 168 - protein, 11 aa, Artificial Sequence
  Synthetic Peptide
RASQGISNYLA

SEQ ID NO: 169 - protein, 15 aa, Artificial Sequence
  Synthetic Peptide
WYQQKPGKAPKLLIY

SEQ ID NO: 170 - protein, 7 aa, Artificial Sequence
  Synthetic Peptide
AASTLQS

SEQ ID NO: 171 - protein, 32 aa, Artificial Sequence; identical to SEQ ID NO: 162
  Synthetic Peptide

SEQ ID NO: 172 - protein, 9 aa, Artificial Sequence
  Synthetic Peptide
QQYYSYPFT

SEQ ID NO: 173 - protein, 10 aa, Artificial Sequence
  Synthetic Peptide
FGPGTKVDIK

SEQ ID NO: 174 - DNA, 324 bp, Artificial Sequence
  Synthetic Polynucleotide
gaagtagtgctgactcaatctccagccaccctgtctgtgtctgcaggggatagagccaccctctcctgcagggccagccagagtgttagccgcgacttagcctggtatcagcagaaacctggccaggctcccaggctcctcatctatggtgcttccaccagggccactgatatcccagtcaggttcagtggcagtgggtctgggacagagttctctctcaccatcagcagcctgcagtctgaagattttgcagtttattactgtcatcaatataaacactggcctcggacgttcggccaggggaccaaggtggaaatcaagcga

SEQ ID NO: 175 - protein, 107 aa, Artificial Sequence
  Synthetic Peptide
EVVLTQSPATLSVSAGDRATLSCRASQSVSRDLAWYQQKPGQAPRLLIYGASTRATDIPVRFSGSGSGTEFSLTISSLQSEDFAVYYCHQYKHWPRTFGQGTKVEIK

SEQ ID NO: 176 - protein, 23 aa, Artificial Sequence
  Synthetic Peptide
EVVLTQSPATLSVSAGDRATLSC

SEQ ID NO: 177 - protein, 11 aa, Artificial Sequence
  Synthetic Peptide
RASQSVSRDLA

SEQ ID NO: 178 - protein, 15 aa, Artificial Sequence
  Synthetic Peptide
WYQQKPGQAPRLLIY

SEQ ID NO: 179 - protein, 7 aa, Artificial Sequence
  Synthetic Peptide
GASTRAT

SEQ ID NO: 180 - protein, 32 aa, Artificial Sequence
  Synthetic Peptide
DIPVRFSGSGSGTEFSLTISSLQSEDFAVYYC

SEQ ID NO: 181 - protein, 9 aa, Artificial Sequence
  Synthetic Peptide
HQYKHWPRT

SEQ ID NO: 182 - protein, 10 aa, Artificial Sequence
  Synthetic Peptide
FGQGTKVEIK

(19) United States
(12) Patent Application Publication
(10) Pub. No.: US 2015/0110794 A1
(43) Pub. Date: Apr. 23, 2015

(54) Anti-Human Cytomegalovirus Antibodies And Use Thereof

(71) Applicant: Cell Signaling Technology, Inc., Danvers, MA (US)

(72) Inventors: Shuji Sato, Somerville, MA (US); Sean Andre Beausoleil, Essex, MA (US); Wan Cheung Cheung, Lexington, MA (US); Roberto D. Polakiewicz, Lexington, MA (US)

(21) Appl. No.: 14/398,074

(22) PCT Filed: Apr. 30, 2013
(86) PCT No.: PCT/US2013/038814

§ 371 Date: Oct. 30, 2014

Related U.S. Application Data
(60) Provisional application No. 61/640,374, filed on Apr. 30, 2012.

(51) Int. Cl. C07K 16/08; G01N 33/569

(52) U.S. Cl. CPC C07K 16/088; G01N 33/56994; C07K 2317/565; C07K 2317/92; C07K 2317/76; G01N 2333/045
USPC 424/139.1; 530/387.9; 536/23.53; 435/320.1; 435/252.3; 435/5; 435/238; 435/252.33; 435/252.35; 435/254.11; 435/254.2; 435/331

(57) ABSTRACT

This disclosure provides anti-human cytomegalovirus antibodies and methods of treatment, prophylaxis, detection, and diagnosis using the same. In another aspect, the disclosure features therapeutic, prophylactic, and/or diagnostic compositions for human cytomegalovirus infection or for a human cytomegalovirus-related disease that include a binding agent (e.g., antibody) or polynucleotide disclosed herein. In some embodiments, the composition is formulated for ocular or topical administration. The compositions can further include one or more human cytomegalovirus-neutralizing antibodies, an intravenous immunoglobulin preparation, and/or one or more antiviral compounds (e.g., ganciclovir, foscamet, cidofovir, or valganciclovir).

### CROSS-REFERENCE TO RELATED APPLICATIONS

This application claims priority to U.S. Provisional Application No. 61/640,374, filed Apr. 30, 2012, the entire contents of which are incorporated herein by reference.

### BACKGROUND

Human cytomegalovirus (hCMV) is a clinically significant pathogen for immunocompromised individuals, solid organ transplant recipients and newborns, who may contract the virus congenitally (Staras et al., 2006, Clin. Infect. Dis., 43:1143-51). Potently neutralizing monoclonal antibodies against hCMV that can be produced consistently in industrial scale batches would have clinical value.

### SUMMARY

This disclosure is based, at least in part, on the isolation of human anti-hCMV antibodies, including antibodies that neutralize hCMV infection activity.

Accordingly, the disclosure features in one aspect anti-human cytomegalovirus envelope glycoprotein B binding agents (e.g., antibodies) that include:

(i) (a) a heavy chain variable region having CDRs 1, 2 and 3 having the amino acid sequences set forth in SEQ ID NOs: 24, 26 and 28, respectively, and (b) a light chain variable region having CDRs 1, 2 and 3 having a set of amino acid sequences set forth in (i) SEQ ID NOs: 69, 71 and 73, respectively; (ii) SEQ ID NOs: 78, 80 and 82, respectively; (iii) SEQ ID NOs: 87, 89 and 91, respectively; (iv) SEQ ID NOs: 96, 98 and 100, respectively; (v) SEQ ID NOs: 105, 107 and 109, respectively; (vi) SEQ ID NOs: 114, 116 and 118, respectively; (vii) SEQ ID NOs: 123, 125 and 127, respectively; (viii) SEQ ID NOs: 132, 134 and 136, respectively; (ix) SEQ ID NOs: 141, 143 and 145, respectively; or (x) SEQ ID NOs: 150, 152 and 154, respectively;

(ii) a heavy chain variable region having CDRs 1, 2 and 3 having the amino acid sequences set forth in SEQ ID NOs: 33, 35 and 37, respectively, and a light chain variable region having CDRs 1, 2 and 3 having the amino acid sequences set forth in SEQ ID NOs: 141, 143 and 145, respectively;

(iii) a heavy chain variable region having CDRs 1, 2 and 3 having the amino acid sequences set forth in SEQ ID NOs: 42, 44 and 46, respectively, and a light chain variable region having CDRs 1, 2 and 3 having the amino acid sequences set forth in SEQ ID NOs: 105, 107 and 109, respectively;

(iv) a heavy chain variable region having CDRs 1, 2 and 3 having the amino acid sequences set forth in SEQ ID NOs: 51, 53 and 55, respectively, and a light chain variable region having CDRs 1, 2 and 3 having the amino acid sequences set forth in SEQ ID NOs: 159, 161 and 163, respectively, or SEQ ID NOs: 168, 170 and 172, respectively;

(v) a heavy chain variable region having CDRs 1, 2 and 3 having the amino acid sequences set forth in SEQ ID NOs: 60, 62 and 64, respectively, and a light chain variable region having CDRs 1, 2 and 3 having the amino acid sequences set forth in SEQ ID NOs: 177, 179 and 181, respectively.

In another aspect, the disclosure features anti-human cytomegalovirus envelope glycoprotein B binding agents (e.g., antibodies) that include a heavy chain variable region having the sequence of SEQ ID NO: 22, 31, 40, 49, or 58 and/or a light chain variable region comprising the sequence of SEQ ID NO: 67, 76, 85, 94, 103, 112, 121, 130, 139, 148, 157, 166, or 175.

In another aspect, the disclosure features anti-human cytomegalovirus envelope glycoprotein B binding agents (e.g., antibodies) that include:

(i) 8 a heavy chain variable region having an amino acid sequence at least 80% identical (e.g., at least 85%, 90%, 95%, 97%, 98%, 99%, or 100% identical) to SEQ ID NO: 22 and a light chain variable region having an amino acid sequence at least 80% identical (e.g., at least 85%, 90%, 95%, 97%, 98%, 99%, or 100% identical) to SEQ ID NO: 67, 76, 85, 94, 103, 112, 121, 130, 139, or 148;

(ii) a heavy chain variable region having an amino acid sequence at least 80% identical (e.g., at least 85%, 90%, 95%, 97%, 98%, 99%, or 100% identical) to SEQ ID NO: 31 and a light chain variable region having an amino acid sequence at least 80% identical (e.g., at least 85%, 90%, 95%, 97%, 98%, 99%, or 100% identical) to SEQ ID NO: 139;

(iii) a heavy chain variable region having an amino acid sequence at least 80% identical (e.g., at least 85%, 90%, 95%, 97%, 98%, 99%, or 100% identical) to SEQ ID NO: 40 and a light chain variable region having an amino acid sequence at least 80% identical (e.g., at least 85%, 90%, 95%, 97%, 98%, 99%, or 100% identical) to SEQ ID NO: 103;

(iv) a heavy chain variable region having an amino acid sequence at least 80% identical (e.g., at least 85%, 90%, 95%, 97%, 98%, 99%, or 100% identical) to SEQ ID NO: 49 and a light chain variable region having an amino acid sequence at least 80% identical (e.g., at least 85%, 90%, 95%, 97%, 98%, 99%, or 100% identical) to SEQ ID NO: 157 or 166;

(v) a heavy chain variable region having an amino acid sequence at least 80% identical (e.g., at least 85%, 90%, 95%, 97%, 98%, 99%, or 100% identical) to SEQ ID NO: 58 and a light chain variable region having an amino acid sequence at least 80% identical (e.g., at least 85%, 90%, 95%, 97%, 98%, 99%, or 100% identical) to SEQ ID NO: 175.

In yet another aspect, the disclosure features anti-human cytomegalovirus envelope glycoprotein B binding agents (e.g., antibodies) that bind to a polypeptide consisting of SEQ ID NO: 2 with a dissociation constant of 1×10⁻⁸ M or less (e.g., 5×10⁻⁹ M or less, 2×10⁻⁹ M or less, 1×10⁻⁹ M or less, or 5×10⁻¹⁰ M or less).

In another aspect, the disclosure features anti-human cytomegalovirus envelope glycoprotein B antibody that binds to a polypeptide consisting of SEQ ID NO: 2 with an association rate of 1×10⁵ M⁻¹s⁻¹ or greater (e.g., 2×10⁵ M⁻¹s⁻¹ or greater, 5×10⁵ M⁻¹s⁻¹ or greater, or 1×10⁶ M⁻¹s⁻¹ or greater) and/or a dissociation rate of 1×10⁻² s⁻¹ or less (e.g., 5×10⁻³ s¹ or less, 2×10⁻³ s⁻¹ or less, 1×10⁻³ s⁻¹ or less, 5×10⁻⁴ s⁻¹ or less, or 2×10⁻⁴ s⁻¹ or less).

In a further aspect, the disclosure features anti-human cytomegalovirus envelope glycoprotein B binding agents (e.g., antibodies), wherein the concentration of binding agent required for 50% inhibition of human cytomegalovirus is 25 ug/ml or less (e.g., 20 ug/ml or less, 10 ug/ml or less, 5 ug/ml or less, 3 ug/ml or less, 2 ug/ml or less, 1 ug/ml or less, 0.5 ug/ml or less, 0.2 ug/ml or less, 0.1 ug/ml or less, or 0.05 ug/ml or less). In some embodiments, the binding agent binds to a polypeptide consisting of SEQ ID NO: 2.

In yet another aspect, the disclosure features anti-human cytomegalovirus envelope glycoprotein B binding agents (e.g., antibodies) that bind to the same epitope as an antibody selected from the group consisting of:

(a) an antibody having a heavy chain amino acid sequence consisting of SEQ ID NO: 3 and a light chain amino acid sequence consisting of SEQ ID NO: 8, 9, 10, 11, 12, 13, 14, 15, 16, or 17;

(b) an antibody having a heavy chain amino acid sequence consisting of SEQ ID NO: 4 and a light chain amino acid sequence consisting of SEQ ID NO: 16;

(c) an antibody having a heavy chain amino acid sequence consisting of SEQ ID NO: 5 and a light chain amino acid sequence consisting of SEQ ID NO: 12;

(d) an antibody having a heavy chain amino acid sequence consisting of SEQ ID NO: 6 and a light chain amino acid sequence consisting of SEQ ID NO: 18 or 19; and

(e) an antibody having a heavy chain amino acid sequence consisting of SEQ ID NO: 7 and a light chain amino acid sequence consisting of SEQ ID NO: 20.

In another aspect, the disclosure features anti-human cytomegalovirus envelope glycoprotein B binding agents (e.g., antibodies) that compete for binding to a polypeptide having an amino acid sequence consisting of SEQ ID NO: 2 with an antibody selected from the group consisting of:

(a) an antibody having a heavy chain amino acid sequence consisting of SEQ ID NO: 3 and a light chain amino acid sequence consisting of SEQ ID NO: 8, 9, 10, 11, 12, 13, 14, 15, 16, or 17;

(b) an antibody having a heavy chain amino acid sequence consisting of SEQ ID NO: 4 and a light chain amino acid sequence consisting of SEQ ID NO: 16;

(c) an antibody having a heavy chain amino acid sequence consisting of SEQ ID NO: 5 and a light chain amino acid sequence consisting of SEQ ID NO: 12;

(d) an antibody having a heavy chain amino acid sequence consisting of SEQ ID NO: 6 and a light chain amino acid sequence consisting of SEQ ID NO: 18 or 19; and

(e) an antibody having a heavy chain amino acid sequence consisting of SEQ ID NO: 7 and a light chain amino acid sequence consisting of SEQ ID NO: 20.

In some embodiments of the above aspects, the binding agent (e.g., antibody) is purified).

In some embodiments of the above aspects, the binding agent (e.g., antibody) is human.

In another aspect, the disclosure features polynucleotides that encode a polypeptide chain (e.g., an antibody heavy or light chain) of any of the above binding agents. For example, the polynucleotide may include a sequence at least 80% identical (e.g., at least 85%, 90%, 95%, 97%, 98%, 99%, or 100% identical) to SEQ ID NO: 21, 30, 39, 48, 57, 66, 75, 84, 93, 102, 111, 120, 129, 138, 147, 156, 165, or 174. The disclosure also features vectors that include the above polynucleotides and isolated cells that include the above polynucleotides and/or vectors. In some embodiments, the disclosure features methods of producing binding agents (e.g., antibodies) that include culturing the isolated cells under conditions where the binding agent is expressed and collecting the binding agent.

In another aspect, the disclosure features methods of detecting a human cytomegalovirus in a sample that include contacting a sample with a binding agent (e.g., antibody) disclosed herein and detecting binding of the antibody to the sample, thereby detecting a human cytomegalovirus in the sample.

In yet another aspect, the disclosure features methods of inhibiting human cytomegalovirus infection of a cell that include contacting the cell with a binding agent (e.g., antibody) disclosed herein.

The disclosure also features the binding agents (e.g., antibodies) disclosed herein and the use thereof for treatment, prophylaxis, or diagnosis of a human cytomegalovirus infection.

In another aspect, the disclosure features therapeutic, prophylactic, and/or diagnostic compositions for human cytomegalovirus infection or for a human cytomegalovirus-related disease that include a binding agent (e.g., antibody) or polynucleotide disclosed herein. In some embodiments, the composition is formulated for ocular or topical administration. The compositions can further include one or more human cytomegalovirus-neutralizing antibodies, an intravenous immunoglobulin preparation, and/or one or more antiviral compounds (e.g., ganciclovir, foscarnet, cidofovir, or valganciclovir).

In a further aspect, the disclosure features methods for treatment of human cytomegalovirus infection, or of a human cytomegalovirus-related disease, that include administering a binding agent (e.g., antibody) disclosed herein to a subject with a human cytomegalovirus infection or human cytomegalovirus-related disease in a therapeutically effective amount. In some embodiments, the subject is immunocompromised or is a pregnant woman. The methods can further include administering to the subject one or more human cytomegalovirus-neutralizing antibodies, an intravenous immunoglobulin preparation, and/or one or more antiviral compounds (e.g., ganciclovir, foscarnet, cidofovir, or valganciclovir).

In yet another aspect, the disclosure features methods for human cytomegalovirus prophylaxis, or human cytomegalovirus-related disease prophylaxis, that include administering a binding agent (e.g., antibody) disclosed herein to a subject in a prophylactically effective amount, wherein the administering results in inhibition or prevention of human cytomegalovirus infection. In some embodiments, the subject is immunocompromised, a pregnant woman, or a transplant patient. In some embodiments, the antibody is administered prior to and/or after exposure to human cytomegalovirus. The methods can further include administering to the subject one or more human cytomegalovirus-neutralizing antibodies, an intravenous immunoglobulin preparation, and/or one or more antiviral compounds (e.g., ganciclovir, foscarnet, cidofovir, or valganciclovir).

In yet another embodiment, the disclosure features methods of diagnosing human cytomegalovirus infection, or human cytomegalovirus-related disease, that include contacting a sample from an individual with a binding agent (e.g., antibody) disclosed herein and detecting binding of the binding agent to human cytomegalovirus, wherein detecting binding of the binding agent to human cytomegalovirus in the sample is indicative of human cytomegalovirus infection in the individual from whom the sample was obtained. In some embodiments, the binding agent is linked to a detectable label.

The compositions disclosed herein can provide monoclonal antibodies against hCMV, which can be produced without the use of blood products. This can provide advantages in ease of production without the possibility of transmitting infectious agents (e.g., viruses or prions). Additionally, the use of monoclonal antibodies can allow for administration of less total active immunoglobulin in a smaller total volume than the use of intravenous immune globulin products, providing for fewer transfusion-related side effects.

Technical and scientific terms used herein have the meaning commonly understood by one of skill in the art to which the present disclosure pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Further aspects, advantages, and embodiments are described in more detail below.

### DETAILED DESCRIPTION

This disclosure describes anti-hCMV binding agents and compositions and methods utilizing the same.

As used herein, by “binding agent” is meant a molecule including, without limitation, an organic molecule such as a polypeptide (e.g., an antibody, as defined herein) or a polynucleotide, or an inorganic molecule such as a small chemical molecule or a synthetic polymer, that is capable of binding to a reference target molecule. In some embodiments, the binding agent specifically binds to the reference target molecule, where the phrase “specifically binds” is defined herein. It shall be understood that the binding agent can specifically bind to an epitope located anywhere on the target molecule. Thus, a binding agent that binds to a fragment of a target molecule necessarily binds the larger target molecule (e.g., a binding agent that specifically binds AD4 of hCMV gB also specifically binds to the entire (i.e., full length) hCMV gB protein).

As used herein, by “specifically binding” or “specifically binds” means that a binding agent (e.g., an antibody) interacts with its target molecule (e.g., hCMV envelope glycoprotein B (gB)), where the interaction is dependent upon the presence of a particular structure (i.e., the antigenic determinant or epitope) on the target molecule; in other words, the reagent is recognizing and binding to a specific structure rather than to all molecules in general. By “binding fragment thereof” means a fragment or portion of a binding reagent (e.g., an antigen binding domain of an antibody) that specifically binds the target molecule. A binding agent that specifically binds to the target molecule may be referred to as a target-specific binding agent. For example, an antibody that specifically binds to a hCMV gB molecule may be referred to as a hCMV gB-specific antibody or an anti-hCMV gB antibody.

By “purified” (or “isolated”) refers to a molecule such as a nucleic acid sequence (e.g., a polynucleotide) or an amino acid sequence (e.g., a polypeptide) that is removed or separated from other components present in its natural environment. For example, an isolated antibody is one that is separated from other components of a eukaryotic cell (e.g., the endoplasmic reticulum or cytoplasmic proteins and RNA). An isolated antibody-encoding polynucleotide is one that is separated from other nuclear components (e.g., histones) and/or from upstream or downstream nucleic acid sequences (e.g., an isolated antibody-encoding polynucleotide may be separated from the endogenous heavy chain or light chain promoter). An isolated nucleic acid sequence or amino acid sequence may be at least 60% free, or at least 75% free, or at least 90% free, or at least 95% free from other components present in natural environment of the indicated nucleic acid sequence or amino acid sequence.

Naturally occurring antibodies (also called immunoglobulins) are made up of two classes of polypeptide chains, light chains and heavy chains. A non-limiting antibody of the disclosure can be an intact, four immunoglobulin chain antibody comprising two heavy chains and two light chains. The heavy chain of the antibody can be of any isotype including IgM, IgG, IgE, IgA or IgD or sub-isotype including IgG1, IgG2, IgG2a, IgG2b, IgG3, IgG4, IgE1, IgE2, etc. The light chain can be a kappa light chain or a lambda light chain. A single naturally occurring antibody comprises two identical copies of a light chain and two identical copies of a heavy chain. The heavy chains, which each contain one variable domain (V_{H}) and multiple constant domains, bind to one another via disulfide bonding within their constant domains to form the “stem” of the antibody. The light chains, which each contain one variable domain (V_{L}) and one constant domain, each bind to one heavy chain via disulfide binding. The variable domain of each light chain is aligned with the variable domain of the heavy chain to which it is bound. The variable regions of both the light chains and heavy chains contain three hypervariable regions sandwiched between four more conserved framework regions (FR). These hypervariable regions, known as the complementary determining regions (CDRs), form loops that comprise the principle antigen binding surface of the antibody (see Kabat, E. A. et al., Sequences of Proteins of Immunological Interest, National Institutes of Health, Bethesda, Md., (1987)). The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding domain.

Thus, as used herein, the term “antibody” as used herein is meant to include intact immunoglobulin molecules (e.g., IgG1, IgG2a, IgG2b, IgG3, IgM, IgD, IgE, IgA) for any species (e.g., human, rodent, camelid), as well as antigen binding domain fragments thereof, such as Fab, Fab′, F(ab′)₂; variants thereof such as scFv, Fv, Fd, dAb, bispecific scFvs, diabodies, linear antibodies (see U.S. Pat. No. 5,641,870; Zapata et al., 1999, Protein Eng., 8:1057-62); single-chain antibody molecules; and multispecific antibodies formed from antibody fragments; and any polypeptide that includes a binding domain which is, or is homologous to, an antibody binding domain. By “antigen binding domain” is meant any portion of an antibody that retains specific binding activity of the intact antibody (i.e., any portion of an antibody that is capable of specific binding to an epitope on the intact antibody's target molecule). An “epitope” is smallest portion of a target molecule capable being specifically bound by the antigen binding domain of an antibody. The minimal size of an epitope may be about five or six to seven amino acids. Non-limiting antigen binding domains include the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody. Antibodies disclosed herein include but are not limited to polyclonal, monoclonal, monospecific, polyspecific antibodies and fragments thereof and chimeric antibodies that include an immunoglobulin binding domain fused to another polypeptide.

In some embodiments, an antibody that specifically binds to a target molecule provide a detection signal at least 5-, 10-, or 20-fold higher than a detection signal provided with other proteins when used in an immunochemical assay. In some embodiments, antibodies that specifically bind to a target molecule do not detect other proteins in immunochemical assays and can immunoprecipitate the target molecule from solution.

In some embodiments an immunoglobulin chain (e.g., a heavy chain or a light chain) may include in order from amino terminus to carboxy terminus a variable region and a constant region. The variable region may include three complementarity determining regions (CDRs), with interspersed framework (FR) regions for a structure FR1, CDR1, FR2, CDR2, FR3, CDR3 and FR4. Also within the disclosure are antibodies that include heavy or light chain variable regions, framework regions and CDRs. The antibody may comprise a heavy chain constant region that comprises some or all of a CH1 region, hinge, CH2 and/or CH3 region. The antibody may comprise a light chain constant region that comprises some or all of a CL region.

Antibodies disclosed herein can be derived from any species of animal, including mammals. Non-limiting exemplary natural antibodies include antibodies derived from human, camelids (e.g., camels and llamas), chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Natural antibodies are the antibodies produced by a host animal. “Genetically altered antibodies” refer to antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

Other antibodies specifically contemplated are oligoclonal antibodies. As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need. Exemplary monoclonal antibodies that can be formulated as oligoclonal antibodies with one or more antibodies disclosed herein are described in U.S. Pat. No. 8,153,129; U.S. Pat. No. 8,124,093; U.S. Pat. No. 7,955,599; US 2012/0093810; US 2012/0020980; US 2011/0305708; US 2004/0082033; US 2008/0213265; and US 2009/0004198.

Recombinant antibodies are also included in the present application. These recombinant antibodies are engineered to have the same amino acid sequence as the natural antibodies or to have altered amino acid sequences of the natural antibodies in the present application. They can be made in any expression systems including both prokaryotic and eukaryotic expression systems or using phage display methods (see, e.g., PCT Publication No. WO91/17271, PCT Publication No. WO92/01047; U.S. Pat. No. 5,969,108; U.S. Pat. No. 6,331,415; U.S. Pat. No. 7,498,024, and U.S. Pat. No. 7,485,291, which are herein incorporated by reference in their entirety).

Antibodies can be engineered in numerous ways. They can be made as single-chain antibodies (including small modular immunopharmaceuticals or SMIPs), Fab and F(ab′)₂ fragments, etc. Antibodies can be humanized, chimerized, deimmunized, or fully human. Numerous publications set forth the many types of antibodies and the methods of engineering such antibodies. For example, see U.S. Patent Publication No. 20060099204; U.S. Pat. Nos. 6,355,245; 6,180,370; 5,693,762; 6,407,213; 6,548,640; 5,565,332; 5,225,539; 6,103,889; and 5,260,203.

The genetically altered antibodies should be functionally equivalent to the above-mentioned natural antibodies. In certain embodiments, modified antibodies provide improved stability or/and therapeutic efficacy. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of this application can be modified post-translationally (e.g., acetylation, and/or phosphorylation) or can be modified synthetically (e.g., the attachment of a labeling group).

Antibodies with engineered or variant constant or Fc regions can be useful in modulating effector functions, such as, for example, antigen-dependent cytotoxicity (ADCC) and complement-dependent cytotoxicity (CDC).

In certain embodiments, genetically altered antibodies are chimeric antibodies and humanized antibodies.

The chimeric antibody is an antibody having portions derived from different antibodies. For example, a chimeric antibody may have a variable region and a constant region derived from two different antibodies. The donor antibodies may be from different species.

The genetically altered antibodies disclosed herein include CDR grafted humanized antibodies. In one embodiment, the humanized antibody comprises heavy and/or light chain CDRs of a non-human donor immunoglobulin and heavy chain and light chain frameworks and constant regions of a human acceptor immunoglobulin. Non-limiting methods for making humanized antibody are disclosed in U.S. Pat. Nos. 5,530,101; 5,585,089; 5,693,761; 5,693,762; and 6,180,370 each of which is incorporated herein by reference in its entirety.

In some embodiments, an antibody disclosed herein will comprise substantially all of at least one, and typically two, variable domains (such as Fab, Fab′, F(ab′)2, Fabc, Fv) in which one or more of the CDR regions are synthetic amino acid sequences that specifically bind to the target molecule, and all or substantially all of the framework regions are those of a human immunoglobulin consensus sequence. The framework regions can also be those of a native human immunoglobulin sequence. Other CDR regions in the antibody can be selected to have human immunoglobulin consensus sequences for such CDRs or the sequence of a native human antibody. The antibody can also comprise at least a portion of an immunoglobulin constant region (Fc) of a human immunoglobulin. Often, an antibody will contain both the light chain as well as at least the variable domain of a heavy chain. The antibody also may include the CH1, hinge, CH2, CH3, and CH4 regions of the heavy chain.

In one embodiment of the disclosure, the antibody fragments are truncated chains (truncated at the carboxyl end). In certain embodiments, these truncated chains possess one or more immunoglobulin activities (e.g., complement fixation activity). Examples of truncated chains include, but are not limited to, Fab fragments (consisting of the VL, VH, CL and CH1 domains); Fd fragments (consisting of the VH and CH1 domains); Fv fragments (consisting of VL and VH domains of a single chain of an antibody); dAb fragments (consisting of a VH domain); isolated CDR regions; (Fab′)₂ fragments, bivalent fragments (comprising two Fab fragments linked by a disulphide bridge at the hinge region). The truncated chains can be produced by conventional biochemical techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CH₁ to produce Fab fragments or after the hinge region to produce (Fab′)₂ fragments. Single chain antibodies may be produced by joining VL- and VH-coding regions with a DNA that encodes a peptide linker connecting the VL and VH protein fragments

“Fv” usually refers to the minimum antibody fragment that contains a complete antigen-recognition and -binding site. This region consists of a dimer of one heavy- and one light-chain variable domain (i.e., a VL domain and a VH domain) in tight, non-covalent association. It is in this configuration that the three CDRs of each variable domain interact to define an antigen-binding site on the surface of the V_{H}-V_{L} dimer. Collectively, the CDRs confer antigen-binding specificity to the antibody. However, even a single variable domain (or half of an Fv comprising three CDRs specific for an antigen) has the ability to recognize and bind antigen, although likely at a lower affinity than the entire binding site. “Single-chain Fv” or “scFv” antibody fragments comprise the V_{H} and V_{L} domains of an antibody, wherein these domains are present in a single polypeptide chain. In certain embodiments, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315.

Papain digestion of an intact antibody produces two identical antigen-binding fragments, called “Fab” fragments, each with a single antigen-binding site, and a residual “Fc” fragment, whose name reflects its ability to crystallize readily. The Fab fragment contains the entire light chain (i.e., the constant domain (CL) and variable domain (VL) of the light chain) together with the first constant domain (CH1) and variable region (VH) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. For example, pepsin treatment of an antibody yields an F(ab′)₂ fragment that has two antigen-combining sites and is still capable of cross-linking antigen. In other words, an F(ab′)₂ fragment comprises two disulfide linked Fab fragments. Other chemical couplings of antibody fragments are also known.

SMIPs are a class of single-chain peptides engineered to include a target binding region and effector domain (CH₂ and CH3 domains). See, e.g., U.S. Patent Application Publication No. 20050238646. The target-binding region may be derived from the variable region or CDRs of an antibody, e.g., an hCMV gB-specific antibody disclosed herein. Alternatively, the target-binding region is derived from a protein that binds the indicated target (e.g., a non-immunoglobulin molecule that binds to hCMV gB).

Bispecific antibodies may be monoclonal, human or humanized antibodies that have binding specificities for at least two different antigens. In the present case, one of the binding specificities is for the indicated target (e.g., hCMV gB), the other one is for any other antigen, such as for example, a cell-surface protein or receptor or receptor subunit. Alternatively, a therapeutic agent may be placed on one arm. The therapeutic agent can be a drug, toxin, enzyme, DNA, radionuclide, etc.

In some embodiments, the antigen-binding fragment can be a diabody. The term “diabody” refers to a small antibody fragment with two antigen-binding sites, which fragment comprises a heavy-chain variable domain (V_{H}) connected to a light-chain variable domain (V_{L}) in the same polypeptide chain (V_{H}-V_{L}). Diabodies can be prepared by constructing scFv fragments with short linkers (about 5-10 residues) between the VH and VL domains such that inter-chain but not intra-chain pairing of the V domains is achieved, resulting in a multivalent fragment, i.e., a fragment having two antigen-binding sites. Since the linker is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

Camelid antibodies refer to a unique type of antibodies that are devoid of light chain, initially discovered from animals of the camelid family. The heavy chains of these so-called heavy-chain antibodies bind their antigen by one single domain, the variable domain of the heavy immunoglobulin chain, referred to as VHH. VHHs show homology with the variable domain of heavy chains of the human VHIII family. The VHHs obtained from an immunized camel, dromedary, or llama have a number of advantages, such as effective production in microorganisms such as Saccharomyces cerevisiae.

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; U.S. Pat. No. 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10: 1455-1460 (1992), regarding primatized antibody. See, e.g., Ladner et al., U.S. Pat. No. 4,946,778; and Bird et al., Science, 242: 423-426 (1988), regarding single chain antibodies.

In addition, functional fragments of antibodies, including fragments of chimeric, humanized, primatized or single chain antibodies, can also be produced. Functional fragments of the subject antibodies retain at least one antigen binding domain function and/or modulation function of the full-length (i.e., intact) antibody from which they are derived. Since the immunoglobulin-related genes contain separate functional regions, each having one or more distinct biological activities, the genes of the antibody fragments may be fused to functional regions from other genes (e.g., enzymes, U.S. Pat. No. 5,004,692, which is incorporated by reference in its entirety) to produce fusion proteins or conjugates having novel properties.

Human antibodies (e.g., those with fully human sequences) may be made by means known in the art, e.g., by phage display using human antibody library sequences or by use of mice genetically engineered to produce antibodies from human gene sequences. Additionally, human antibodies may be derived from antibodies or cells in circulation, e.g., using the methods described in WO 2010/011337 and/or U.S. application Ser. No. 13/416,582.

Non-immunoglobulin binding polypeptides are also contemplated. For example, CDRs from an antibody disclosed herein may be inserted into a suitable non-immunoglobulin scaffold to create a non-immunoglobulin binding polypeptide. Suitable candidate scaffold structures may be derived from, for example, members of fibronectin type III and cadherin superfamilies.

Methods for identifying the CDR regions of an antibody by analyzing the amino acid sequence of the antibody are well known (see, e.g., Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007)).

Also contemplated are other equivalent non-antibody molecules, such as protein binding domains or aptamers, which specifically bind to a target molecule described herein (e.g., the hCMV gB). See, e.g., Neuberger et al., Nature 312: 604 (1984). Aptamers are oligonucleic acid or peptide molecules that bind a specific target molecule. DNA or RNA aptamers are typically short oligonucleotides, engineered through repeated rounds of selection to bind to a molecular target. Peptide aptamers typically consist of a variable peptide loop attached at both ends to a protein scaffold. This double structural constraint generally increases the binding affinity of the peptide aptamer to levels comparable to an antibody (nanomolar range).

In various embodiments, the binding agent is an antibody that specifically binds to a hCMV gB molecule. In some embodiments, the binding agent is an antibody having one or more polypeptide sequences selected from any one of SEQ ID NOs: 3-20, 22-29, 31-38, 40-47, 49-56, 58-65, 67-74, 76-83, 85-92, 94-101, 103-110, 112-119, 121-128, 130-137, 139-146, 148-155, 157-164, 166-173, and 175-182. In various embodiments, the binding agent includes at least one complementary determining region (CDR), wherein the CDR includes a sequence selected from any one of SEQ ID NOs: 24, 26, 28, 33, 35, 37, 42, 44, 46, 51, 53, 55, 60, 62, 64, 69, 71, 73, 78, 80, 82, 87, 89, 91, 96, 98, 100, 105, 107, 109, 114, 116, 118, 123, 125, 127, 132, 134, 136, 141, 143, 145, 150, 152, 154, 159, 161, 163, 168, 170, 172, 177, 179, and 181. In various embodiments, the binding agent includes at least one variable region that includes a sequence selected from any one of SEQ ID NOs: 22, 31, 40, 49, 58, 67, 76, 85, 94, 103, 112, 121, 130, 139, 148, 157, 166, and 175.

In further embodiments, the binding agent specifically binds to an epitope within SEQ ID NO: 2.

The binding agents of the present disclosure include the antibodies having the amino acid sequences set forth herein (whether or not including a leader sequence), and binding agents that may include at least six contiguous amino acids encompassing the amino acid sequence of one or more CDR domains (either from the heavy chain or the light chain, or both) disclosed herein, as well as polypeptides that are at least 80% identical, or at least 85% identical, or at least 90%, 95%, 96%, 97%, 98% or 99% identical to those described above (e.g., at least 80% identical, at least 85% identical, at least 90% identical, or at least 95% identical, or at least 96%, 97%, 98% or 99% identical to any one of SEQ ID NOs: 3-20, 22, 31, 40, 49, 58, 67, 76, 85, 94, 103, 112, 121, 130, 139, 148, 157, 166, or 175).

By “% identical” (or “% identity”) for two polypeptides or two polynucleotides is intended a similarity score produced by comparing the amino acid sequences of the two polypeptides or by comparing the nucleotides sequences of the two polynucleotides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2: 482-489 (1981)) to find the best segment of similarity between two sequences.

General techniques for measuring the affinity of an antibody for an antigen include ELISA, RIA, and surface plasmon resonance. Kinetic parameters, such as dissociation constant, on rate, and off rate, may be measured by surface plasmon resonance using, e.g., a BIAcore sensor.

As used herein, the terms “polypeptide”, “peptide” and “protein” are used interchangeably herein to refer to polymers of amino acids of any length. The polymer may be linear or branched, and it may comprise modified amino acids. Where the amino acid sequence is provided, unless otherwise specified, the sequence is in an N-terminal to C-terminal orientation. In some embodiments, the polymer may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides disclosed herein are based upon antibodies, the polypeptides can occur as single chains or associated chains.

The terms “polynucleotide,” “nucleic acid molecule,” and “nucleic acid sequence” are used interchangeably herein to refer to polymers of nucleotides of any length, and include, without limitation, DNA, RNA, DNA/RNA hybrids, and modifications thereof. Unless otherwise specified, where the nucleotide sequence is provided, the nucleotides are set forth in a 5′ to 3′ orientation. Thus, the nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The present application also provides the polynucleotide molecules encoding analogs of the binding agents (e.g., antibodies) described herein. Because of the degeneracy of the genetic code, a number of different nucleic acid sequences may encode each antibody amino acid sequence. The desired nucleic acid sequences can be produced by de novo solid-phase DNA synthesis or by PCR mutagenesis of an earlier prepared variant of the desired polynucleotide. In one embodiment, the codons that are used comprise those that are typical for human, rabbit, or mouse (see, e.g., Nakamura, Y., Nucleic Acids Res. 28: 292 (2000)).

In additional, the present disclosure provides, in part, isolated polynucleotides that encode a binding agent disclosed herein, nucleotide probes that hybridize to such polynucleotides, and methods, vectors, and host cells for utilizing such polynucleotides to produce recombinant fusion polypeptides.

Some nucleotide sequences and polypeptide sequences disclosed herein may have been determined using an automated peptide sequencer. As is known in the art for any DNA sequence determined by this automated approach, any nucleotide sequence determined herein may contain some errors. Nucleotide sequences determined by automation are typically at least about 90% identical, and more typically at least about 95% to about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. Unless otherwise indicated, each nucleotide sequence set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having a sequence disclosed herein is intended to indicate an RNA molecule having a sequence in which each deoxyribonucleotide A, G or C of the sequence has been replaced by the corresponding ribonucleotide A, G or C, and each deoxyribonucleotide T has been replaced by a ribonucleotide U.

In some embodiments, the disclosure provides isolated polynucleotides (and isolated polynucleotides complementary thereto) that include a nucleotide sequence at least about 80% identical (e.g., at least about 85%, 90%, 95%, 97%, 98%, 99%, or 100% identical) to the sequence of any one of SEQ ID NOs: 21, 30, 39, 48, 57, 66, 75, 84, 93, 102, 111, 120, 129, 138, 147, 156, 165, or 174. In some embodiments, the disclosure provides an isolated polynucleotide (or an isolated polynucleotide complementary thereto) that includes a nucleotide sequence at least about 80% identical (e.g., at least about 85%, 90%, 95%, 97%, 98%, 99%, or 100% identical) identical to nucleotide sequence encoding an antibody (or fragment thereof) comprising an amino acid sequence disclosed herein.

Using the information provided herein, such as the nucleotide sequences set forth in SEQ ID NOs: 21, 30, 39, 48, 57, 66, 75, 84, 93, 102, 111, 120, 129, 138, 147, 156, 165, or 174, a nucleic acid molecule encoding a polypeptide binding agent (e.g., an antibody) as disclosed herein may be obtained using standard cloning and screening procedures, such as those for cloning cDNAs using mRNA as starting material.

As indicated, the present disclosure provides, in part, full-length antibodies. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present disclosure provides, in part, nucleotide sequences encoding a heavy or light chain that includes any one of SEQ ID NOs: 3-20, with additional nucleic acid residues located 5′ to the 5′-terminal residues of the coding sequence. Likewise, the disclosure provides nucleotide sequences encoding CDRs, with additional nucleic acid residues located 5′ to the 5′-terminal residues of a polynucleotide that encodes a variable region disclosed herein (e.g., a variable region including the sequence set forth in any one of SEQ ID NOs: 40, 49, 58, 67, 76, 85, 94, 103, 112, 121, 130, 139, 148, 157, 166, and 175) and/or CDR disclosed herein (e.g., a CDR comprising the amino acid sequence set forth in any one of SEQ ID NOs: 24, 26, 28, 33, 35, 37, 42, 44, 46, 51, 53, 55, 60, 62, 64, 69, 71, 73, 78, 80, 82, 87, 89, 91, 96, 98, 100, 105, 107, 109, 114, 116, 118, 123, 125, 127, 132, 134, 136, 141, 143, 145, 150, 152, 154, 159, 161, 163, 168, 170, 172, 177, 179, and 181).

In some embodiments, the antibody-encoding or binding agent-encoding polynucleotide comprises the nucleotide sequence set forth in SEQ ID NOs: 21, 30, 39, 48, 57, 66, 75, 84, 93, 102, 111, 120, 129, 138, 147, 156, 165, or 174. In some embodiments, the antibody-encoding or binding agent-encoding polynucleotide comprises a nucleotide sequence that encodes a variable region having the amino acid sequence set forth in any one of SEQ ID NOs: 22, 31, 40, 49, 58, 67, 76, 85, 94, 103, 112, 121, 130, 139, 148, 157, 166, and 175 and/or a CDR having the amino acid sequence set forth in any one of SEQ ID NOs: 24, 26, 28, 33, 35, 37, 42, 44, 46, 51, 53, 55, 60, 62, 64, 69, 71, 73, 78, 80, 82, 87, 89, 91, 96, 98, 100, 105, 107, 109, 114, 116, 118, 123, 125, 127, 132, 134, 136, 141, 143, 145, 150, 152, 154, 159, 161, 163, 168, 170, 172, 177, 179, and 181.

In some embodiments, the polynucleotide encodes a polypeptide having the amino acid sequence set forth in any one of SEQ ID NOs: 3-20.

As indicated, polynucleotides of the present disclosure may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

Isolated polynucleotides of the disclosure may be nucleic acid molecules, DNA or RNA, which have been removed from their native environment. For example, recombinant DNA molecules contained in a vector are considered isolated for the purposes of the present disclosure. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present disclosure. Isolated nucleic acid molecules according to the present disclosure further include such molecules produced synthetically.

Polynucleotides of the disclosure include the nucleic acid molecules having the sequences set forth in SEQ ID NOs: 21, 30, 39, 48, 57, 66, 75, 84, 93, 102, 111, 120, 129, 138, 147, 156, 165, or 174, nucleic acid molecules comprising the coding sequence for the antibodies and binding agents of the disclosure that include a sequence different from those described above but which, due to the degeneracy of the genetic code, still encode an antibody or binding agent disclosed herein. The genetic code is well known in the art. Thus, it would be routine for one skilled in the art to generate such degenerate variants.

The disclosure further provides isolated polynucleotides comprising nucleotide sequences having a sequence complementary to one of the binding agent-encoding or antibody-encoding polynucleotides disclosed herein. Such isolated molecules, particularly DNA molecules, are useful as probes for gene mapping, by in situ hybridization with chromosomes, and for detecting expression of the antibody in tissue (e.g., human tissue), for instance, by northern blot analysis.

In some embodiments, the binding agents (e.g., antibodies) of the disclosure are encoded by at least a portion of the nucleotide sequences set forth herein. As used herein, a “portion” or “fragment” means a sequence fragment comprising a number of contiguous amino acid residues (if a polypeptide fragment (which may also be referred to herein a peptide)) or a sequence fragment comprising a number of nucleotide residues (if a polynucleotide fragment) that is less than the number of such residues in the whole sequence (e.g., a 50 nucleotide sequence is a portion of a 100 nucleotide long sequence). In other words, fragment of an indicated molecule that is smaller than the indicated molecule. For example, the binding agent-encoding polynucleotides and/or the antibody-encoding polynucleotides disclosed herein may comprise portions of intron sequences that do not encode any amino acids in the resulting binding agent or antibody. A fragment of a polynucleotide may be at least about 15 nucleotides, or at least about 20 nucleotides, or at least about 30 nucleotides, or at least about 40 nucleotides in length, which are useful as diagnostic probes and primers as discussed herein. Of course, larger fragments of about 50-1500 nucleotides in length are also useful according to the present disclosure, as are fragments corresponding to most, if not all, of the antibody-encoding or binding agent-encoding nucleotide sequence of the cDNAs having sequences set forth in SEQ ID NOs: 21, 30, 39, 48, 57, 66, 75, 84, 93, 102, 111, 120, 129, 138, 147, 156, 165, or 174. By “a fragment at least 20 nucleotides in length”, for example, is meant fragments that include 20 or more contiguous nucleotides from the respective nucleotide sequences from which the fragments are derived.

Polynucleotide fragments are useful as nucleotide probes for use diagnostically according to conventional DNA hybridization techniques or for use as primers for amplification of a target sequence by the polymerase chain reaction (PCR), as described, for instance, in MOLECULAR CLONING, A LABORATORY MANUAL, 2nd. edition, Sambrook, J., Fritsch, E. F. and Maniatis, T., eds., Cold Spring Harbor Laboratory Press, Cold Spring Harbor, N.Y. (1989), the entire disclosure of which is hereby incorporated herein by reference. Of course, a polynucleotide which hybridizes only to a poly A sequence or to a complementary stretch of T (or U) resides, would not be included in a polynucleotide of the disclosure used to hybridize to a portion of a nucleic acid disclosed herein, since such a polynucleotide would hybridize to any nucleic acid molecule containing a poly (A) stretch or the complement thereof (e.g., practically any double-stranded cDNA clone). Generation of such DNA fragments is routine to the skilled artisan, and may be accomplished, by way of example, by restriction endonuclease cleavage or shearing by sonication of DNA obtainable from the cDNA clone described herein or synthesized according to the sequence disclosed herein. Alternatively, such fragments can be directly generated synthetically.

In another aspect, the disclosure provides an isolated polynucleotide (e.g., a nucleotide probe) that hybridizes under stringent conditions to a binding agent-encoding or a antibody-encoding polynucleotide disclosed herein (e.g., any one of SEQ ID NOs: 21, 30, 39, 48, 57, 66, 75, 84, 93, 102, 111, 120, 129, 138, 147, 156, 165, or 174). The term “stringent conditions” with respect to nucleotide sequence or nucleotide probe hybridization conditions is the “stringency” that occurs within a range from about Tₘ minus 5° C. (i.e., 5° C. below the melting temperature (Tₘ) of the probe or sequence) to about 20° C. to 25° C. below Tₘ. Typical stringent conditions are: overnight incubation at 42° C. in a solution comprising: 50% formamide, 5×.SSC (750 mM NaCl, 75 mM trisodium citrate), 50 mM sodium phosphate (pH 7.6), 5×Denhardt's solution, 10% dextran sulfate, and 20 micrograms/ml denatured, sheared salmon sperm DNA, followed by washing the filters in 0.1×SSC at about 65° C. As will be understood by those of skill in the art, the stringency of hybridization may be altered in order to identify or detect identical or related polynucleotide sequences.

By a polynucleotide or nucleotide probe that hybridizes to a reference polynucleotide is intended that the polynucleotide or nucleotide probe (e.g., DNA, RNA, or a DNA-RNA hybrid) hybridizes along the entire length of the reference polynucleotide or hybridizes to a portion of the reference polynucleotide that is at least about 15 nucleotides (nt), or to at least about 20 nt, or to at least about 30 nt, or to about 30-70 nt of the reference polynucleotide. These nucleotide probes of the disclosure are useful as diagnostic probes and primers (e.g. for PCR) as discussed herein.

Of course, polynucleotides hybridizing to a larger portion of the reference polynucleotide, for instance, a portion 50-750 nt in length, or even to the entire length of the reference polynucleotide, are useful as probes according to the present disclosure, as are polynucleotides corresponding to most, if not all, of the nucleotide sequence of the cDNAs described herein or the nucleotide sequences set forth in SEQ ID NOs: 21, 30, 39, 48, 57, 66, 75, 84, 93, 102, 111, 120, 129, 138, 147, 156, 165, and 174.

As indicated, nucleic acid molecules of the present disclosure, which encode binding agents disclosed herein, may include but are not limited to those encoding the amino acid sequence of the mature intact polypeptide, by itself; fragments thereof; the coding sequence for the mature polypeptide and additional sequences, such as those encoding the leader or secretory sequence, such as a pre-, or pro- or pre-pro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide that facilitates purification of the fused polypeptide. In certain embodiments of this aspect of the disclosure, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86: 821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the binding agents and/or antibodies of the disclosure fused to an Fc domain at the N- or C-terminus.

The present disclosure further relates to variants of the nucleic acid molecules disclosed herein, which encode portions, analogs or derivatives of a binding agent or antibody disclosed herein. Variants may occur naturally, such as a natural allelic variant. By an “allelic variant” is intended one of several alternate forms of a gene occupying a given locus on a chromosome of an organism. See, e.g. GENES II, Lewin, B., ed., John Wiley & Sons, New York (1985). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Some alterations included in the disclosure are silent substitutions, additions and deletions, which do not alter the properties and activities (e.g. specific binding activity) of the binding agent and/or antibody disclosed herein.

Further embodiments of the disclosure include isolated polynucleotides comprising a nucleotide sequence at least 80% identical, e.g., at least 85%, 90%, 95%, 96%, 97%, 98% or 99% identical, to a binding agent-encoding or antibody-encoding polynucleotide of the disclosure.

As a practical matter, whether any particular nucleic acid molecule is at least 80%, 85%, 90%, 95%, 96%, 97%, 98% or 99% identical to, for example, the nucleotide sequences set forth in SEQ ID NOs: 21, 30, 39, 48, 57, 66, 75, 84, 93, 102, 111, 120, 129, 138, 147, 156, 165, or 174 or to a nucleotide sequence that encodes a polypeptide disclosed herein can be determined conventionally using known computer programs such as the Bestfit program (Wisconsin Sequence Analysis Package, Version 8 for Unix, Genetics Computer Group, University Research Park, 575 Science Drive, Madison, Wis. 53711.

Due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the cDNAs described herein, to the nucleic acid sequences set forth in SEQ ID NOs: 21, 30, 39, 48, 57, 66, 75, 84, 93, 102, 111, 120, 129, 138, 147, 156, 165, or 174 or to nucleic acid sequences encoding a polypeptide disclosed herein will encode a polypeptide having specific binding activity (e.g., in combination with a cognate heavy or light chain). In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide that retains the specific binding activity of the reference binding agent or antibody of the disclosure. This is because the skilled artisan is fully aware of amino acid substitutions that are either less likely or not likely to significantly effect protein function (e.g., replacing one aliphatic amino acid with a second aliphatic amino acid). For example, guidance concerning how to make phenotypically silent amino acid substitutions is provided in Bowie et al., “Deciphering the Message in Protein Sequences: Tolerance to Amino Acid Substitutions,” Science 247: 1306-1310 (1990), which describes two main approaches for studying the tolerance of an amino acid sequence to change. Skilled artisans familiar with such techniques also appreciate which amino acid changes are likely to be permissive at a certain position of the protein. For example, most buried amino acid residues require nonpolar side chains, whereas few features of surface side chains are generally conserved. Other such phenotypically silent substitutions are described in Bowie et al., supra., and the references cited therein.

Methods for DNA sequencing that are well known and generally available in the art may be used to practice any polynucleotide embodiments of the disclosure. The methods may employ such enzymes as the Klenow fragment of DNA polymerase I, SEQUENASE (US Biochemical Corp, Cleveland, Ohio), Taq polymerase (Invitrogen), thermostable T7 polymerase (Amersham, Chicago, Ill.), or combinations of recombinant polymerases and proofreading exonucleases such as the ELONGASE Amplification System marketed by Gibco BRL (Gaithersburg, Md.). The process may be automated with machines such as the Hamilton Micro Lab 2200 (Hamilton, Reno, Nev.), Peltier Thermal Cycler (PTC200; MJ Research, Watertown, Mass.), ABI 377 DNA sequencers (Applied Biosystems), and 454 sequencers (Roche).

Polynucleotide sequences encoding a binding agent or antibody disclosed herein may be extended utilizing a partial nucleotide sequence and employing various methods known in the art to detect upstream sequences such as promoters and regulatory elements. For example, one method that may be employed, “restriction-site” PCR, uses universal primers to retrieve unknown sequence adjacent to a known locus (Sarkar, G., PCR Methods Applic. 2: 318-322 (1993)). In particular, genomic DNA is first amplified in the presence of primer to linker sequence and a primer specific to the known region. Exemplary primers are those described in Example 4 herein. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia et al., Nucleic Acids Res. 16: 8186 (1988)). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. Another method which may be used to retrieve unknown sequences is that described in Parker et al., 1991, Nucleic Acids Res., 19:3055-60). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

When screening for full-length cDNAs, libraries that have been size-selected to include larger cDNAs may be used or random-primed libraries, which contain more sequences that contain the 5′ regions of genes. A randomly primed library is useful for situations in which an oligo d(T) library does not yield a full-length cDNA. Genomic libraries may be useful for extension of sequence into the 5′ and 3′ non-transcribed regulatory regions.

Capillary electrophoresis systems, which are commercially available, may be used to analyze the size or confirm the nucleotide sequence of sequencing or PCR products. In particular, capillary sequencing may employ flowable polymers for electrophoretic separation, four different fluorescent dyes (one for each nucleotide) that are laser activated, and detection of the emitted wavelengths by a charge coupled device camera. Output/light intensity may be converted to electrical signal using appropriate software (e.g., GENOTYPER and SEQUENCE NAVIGATOR, Applied Biosystems) and the entire process from loading of samples to computer analysis and electronic data display may be computer controlled. Capillary electrophoresis is useful for the sequencing of small pieces of DNA that might be present in limited amounts in a particular sample.

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant binding agent polypeptides (e.g., antibodies) or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as encoded polypeptide in a host cell introduced with the expression vector. Thus, in an expression vector, the polynucleotide of interest is positioned for expression in the vector by being operably linked with regulatory elements such as a promoter, enhancer, poly-A tail, etc., either within the vector or in the genome of the host cell at or near or flanking the integration site of the polynucleotide of interest such that the polynucleotide of interest will be translated in the host cell introduced with the expression vector. By “introduced” is meant that a vector is inserted into the host cell by any means including, without limitation, electroporation, fusion with a vector-containing liposomes, chemical transfection (e.g., DEAE-dextran), transformation, transvection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some embodiments, a polynucleotide disclosed herein (e.g., a polynucleotide that encodes a polypeptide disclosed herein) may be introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad. Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques 6:616-627, 1988; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art. The DNA may also be “naked,” as described, for example, in Ulmer et al., 1993, Science 259:1745-1749, and reviewed by Cohen, 1993, Science 259:1691-1692. The uptake of naked DNA may be increased by coating the DNA onto biodegradable beads, which are efficiently transported into the cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. The methods disclosed herein may be practiced with vectors comprising cis-acting control regions to the polynucleotide of interest. Appropriate trans-acting factors may be supplied by the host, supplied by a complementing vector or supplied by the vector itself upon introduction into the host. In certain embodiments in this regard, the vectors provide for specific expression, which may be inducible and/or cell type-specific (e.g., those inducible by environmental factors that are easy to manipulate, such as temperature and nutrient additives).

For expression he DNA insert comprising an antibody-encoding or polypeptide-encoding polynucleotide disclosed herein may be operatively linked to an appropriate promoter, such as the phage lambda PL promoter, the E. coli lac, tip and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA or UAG) appropriately positioned at the end of the polypeptide to be translated.

As indicated, the expression vectors may include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the above-described host cells are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) CURRENT PROTOCOLS IN MOLECULAR BIOLOGY, John Wiley & Sons, New York, N.Y, and Grant et al., Methods Enzymol. 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., BASIC METHODS IN MOLECULAR BIOLOGY (1986).

Transcription of DNA encoding a binding agent or antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at basepairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., binding agent or antibody) may be expressed in a modified form, such as a fusion protein (e.g., a GST-fusion), and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

In one non-limiting example, a binding agent or antibody of the disclosure may comprise a heterologous region from an immunoglobulin that is useful to solubilize proteins. For example, U.S. Pat. No. 7,253,264 discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties.

The binding agents and antibodies can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. In some embodiments, high performance liquid chromatography (“HPLC”) is employed for purification. Polypeptides of the present disclosure include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present disclosure may be glycosylated or may be non-glycosylated. In addition, polypeptides of the disclosure may also include an initial modified methionine residue, in some cases as a result of host-mediated processes.

Accordingly, in another embodiment, the disclosure provides a method for producing a recombinant binding agent or antibody by culturing a recombinant host cell (as described above) under conditions suitable for the expression of the fusion polypeptide and recovering the polypeptide. Culture conditions suitable for the growth of host cells and the expression of recombinant polypeptides from such cells are well known to those of skill in the art. See, e.g., CURRENT PROTOCOLS IN MOLECULAR BIOLOGY, Ausubel F M et al., eds., Volume 2, Chapter 16, Wiley Interscience.

The disclosure also provides immortalized cell lines that produce an antibody disclosed herein. For example, hybridoma clones, constructed as described above, that produce monoclonal antibodies to the target molecule (e.g., hCMV gB) disclosed herein are also provided. Similarly, the disclosure includes recombinant cells producing an antibody as disclosed herein, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.).

The disclosure also provides binding agents, particularly antibodies that specifically bind to an epitope on a target molecule. Likewise, the disclosure provides epitopes useful for identifying the binding agents that specifically bind to a target molecule comprising the epitope.

Epitope mapping can be done using standard methods. For example, phage display is an in vitro selection technique in which a peptide is genetically fused to a coat protein of a bacteriophage resulting in display of a fused protein on the exterior of the virion. Biopanning of these virions by incubating the pool of phage displayed variants with a specific antibody of interest, which has been immobilized on a plate. The unbound phage is then washed away and the specifically bound phage is then eluted. The eluted phage is then amplified in E. coli and the process is repeated, resulting in enrichment of the phage pool in favor of the tightest binding sequences.

An advantage of this technology is that it allows for the screening of greater than 10⁹ sequences in an unbiased way. Phage display is especially useful if the immunogen is unknown or a large protein fragment.

One of the limitations to phage display includes cross contamination between phage particles. Cross contamination between phage particles may enrich for sequences that do not specifically bind the antibody. Additionally, sequences that are not found in nature will be present in the phage displayed peptide library. These sequences may not resemble the immunizing peptide at all and may bind tightly to the antibody of interest. Retrieving sequences that do not resemble the immunizing peptide can be very confounding and it is difficult to decipher whether these peptides are contamination or unnatural peptides with high binding affinity to the antibody of interest.

The binding agents of the present disclosure may be employed in various methods. For example, the binding agents of the disclosure may be used in any known assay method, such competitive binding assays, direct and indirect sandwich assays, and immunoprecipitation assays. Zola, Monoclonal Antibodies: A Manual of Techniques, pp. 147-158 (CRC Press, Inc. 1987). For use in such methods (e.g., for use in in vitro assays), the binding agents may be detectably labeled (e.g., with a fluorophore such as FITC or phycoerythrin or with an enzyme substrate, such as a substrate for horse radish peroxidase) for easy detection. As discussed below, the binding agents of the disclosure may be used for in vivo diagnostic assays, such as in vivo imaging. In some embodiments, the antibody is labeled with a radionucleotide (such as ³H, ¹¹¹In, ¹⁴C, ³²P, ¹²³I) so that the cells or tissue of interest can be localized using immunoscintiography. Methods of conjugating labels to a binding agent (such as an antibody) are known in the art. In other embodiments of the disclosure, binding agents disclosed herein need not be labeled, and the presence thereof can be detected using a labeled antibody, which binds to the binding agent.

The methods of detection and diagnosis may be performed on any biological sample, e.g., a sample from an individual infected with hCMV or suspected of being infected with hCMV. The biological sample can be a bodily fluid (e.g., urine, saliva, blood, tears, semen, or breast milk) or a sample that includes cells.

The antibody may also be used as staining reagent in pathology, following techniques well known in the art.

In another aspect, the disclosure provides methods for detecting hCMV. The methods include contacting a sample suspected of containing hCMV with a binding agent disclosed herein and detecting specific binding of the binding agent to the sample, wherein presence of specific binding of the binding agent to the sample identifies the sample as containing hCMV.

Such detection of specific binding by the binding agent to the sample (e.g., detection of a binding agent:sample complex) may be made by any known method including, without limitation, western blotting analysis, immunohistochemistry (IHC) analysis, immunofluorescence (IF) analysis, flow cytometry analysis, FACS analysis, ELISA, and immunoprecipitation. See, generally, Immunological Methods, Vols. I and II (Lefkovits and Pernis, eds., Academic Press, NY, 1979 and 1981, herein incorporated by reference.

As used herein, an “individual,” also referred to herein as a “subject,” or “patient” is a vertebrate animal, such a mammal (e.g., a human). Mammals include, without limitation, to, farm animals (such as cows, pigs, and chicken), domestic animals (such as cats, parrots, turtles, lizards, dogs, and horses), primates (such as chimpanzees and gorillas), and rodents (such as mice and rats). The patient may or may not be afflicted with a condition (e.g., a CMV infection) and/or may or may not presently show symptoms. In some embodiments, the subject is infected with CMV (e.g., hCMV). In some embodiments, the subject is at risk for CMV (e.g., hCMV) infection. In some embodiments, the subject is undergoing or has undergone additional treatment (e.g., treatment with an intravenous immunoglobulin preparation, a different anti-CMV antibody, or an antiviral compound).

As used herein, the term “biological sample” is used in its broadest sense, and means any biological sample suspected of containing a molecule of interest, and may comprise a cell, an extract from cells, blood, urine, marrow, or a tissue, and the like. The biological sample can be a bodily fluid (e.g., urine, saliva, blood, tears, semen, or breast milk) or a sample that includes cells (e.g., peripheral blood leukocytes). Biological samples useful in the practice of the methods of the disclosure may be obtained from any mammal in which hCMV infection might be present.

Cellular extracts of the foregoing biological samples may be prepared, either crude or partially (or entirely) purified, in accordance with standard techniques, and used in the methods of the disclosure. Alternatively, biological samples comprising whole cells may be utilized in assay formats such as immunohistochemistry (IHC), flow cytometry (FC), and immunofluorescence (IF).

The neutralization activity of binding agents can be characterized using the cell and/or animal models available for gB, as shown in the literature using panels of human sera (Navarro et al., 1997, J. Med. Virol., 52:451-459) or of murine monoclonal antibodies (Schoppel K et al., 1996, Virology, 216:133-145), possibly in combination with ELISA or western blot using hCMV-specific truncated proteins or synthetic peptides.

As used herein, by an “effective amount” is an amount or dosage sufficient to effect beneficial or desired results including halting, slowing, halting, retarding, or inhibiting progression of an hCMV infection in a patient or preventing or inhibiting development of hCMV infection in a patient. An effective amount will vary depending upon, e.g., an age and a body weight of a subject to which the a binding agent, binding agent-encoding polynucleotide, vector comprising the polynucleotide and/or compositions thereof is to be administered, a severity of symptoms and a route of administration, and thus administration is determined on an individual basis. In general, the daily adult dosage for oral administration is about 0.1 to 1000 mg, given as a single dose or in divided doses. For continuous intravenous administration, the compositions can be administered in the range of 0.01 μg/kg/min to 1.0 μg/kg/min, desirably 0.025 μg/kg/min to 0.1 μg/kg/min.

An effective amount can be administered in one or more administrations. By way of example, an effective amount of a binding agent is an amount sufficient to ameliorate, stop, stabilize, reverse, inhibit, slow and/or delay progression of an hCMV infection condition or an hCMV-related disease in a patient or is an amount sufficient to ameliorate, stop, stabilize, reverse, slow and/or delay hCMV infection of a cell (e.g., a biospsied cell) in vitro. As is understood in the art, an effective amount of a binding agent may vary, depending on, inter alia, patient history as well as other factors such as the type (and/or dosage) of binding agent used.

Effective amounts and schedules for administering the binding agents, binding agent-encoding polynucleotides, and/or compositions disclosed herein may be determined empirically, and making such determinations is within the skill in the art. Those skilled in the art will understand that the dosage that must be administered will vary depending on, for example, the mammal that will receive the binding agents, binding agent-encoding polynucleotides, and/or compositions disclosed herein, the route of administration, the particular type of binding agents, binding agent-encoding polynucleotides, and/or compositions disclosed herein used and other drugs being administered to the mammal. Where the patient is administered an antibody and/or a composition comprising an antibody, guidance in selecting appropriate doses for antibody is found in the literature on therapeutic uses of antibodies, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of a binding agent used alone might range from about 1 μg/kg to up to 100 mg/kg of body weight or more per day, depending on the factors mentioned above. Generally, any of the following doses may be used: a dose of at least about 50 mg/kg body weight; at least about 10 mg/kg body weight; at least about 3 mg/kg body weight; at least about 1 mg/kg body weight; at least about 750 μg/kg body weight; at least about 500 μg/kg body weight; at least about 250 μg/kg body weight; at least about 100 μg/kg body weight; at least about 50 μg/kg body weight; at least about 10 μg/kg body weight; at least about 1 μg/kg body weight, or more, is administered. In some embodiments, a dose of a binding agent (e.g., antibody) provided herein is between about 0.01 mg/kg and about 50 mg/kg, between about 0.05 mg/kg and about 40 mg/kg, between about 0.1 mg and about 30 mg/kg, between about 0.1 mg and about 20 mg/kg, between about 0.5 mg and about 15 mg, or between about 1 mg and 10 mg. In some embodiments, the dose is between about 1 mg and 5 mg. In some alternative embodiments, the dose is between about 5 mg and 10 mg.

In some embodiments, the methods described herein further comprise the step of treating the subject with an additional form of therapy. In some embodiments, the additional form of therapy is an additional anti-viral composition. In some embodiments the methods described herein further comprise the step of treating the subject with an intravenous immunoglobulin preparation, a different anti-hCMV antibody (e.g., an antibody against hCMV gH, gB, or UL128 and UL130 proteins), and/or an antiviral compound. In some embodiments, the antiviral compound is ganciclovir, foscarnet, cidofovir, or valganciclovir.

The methods described herein (including therapeutic methods) can be accomplished by a single direct injection or infusion at a single time point or multiple time points to a single or multiple sites. Administration can also be nearly simultaneous to multiple sites. Frequency of administration may be determined and adjusted over the course of therapy, and is base on accomplishing desired results. In some cases, sustained continuous release formulations of binding agents (including antibodies), polynucleotides, and pharmaceutical compositions disclosed herein may be appropriate. Various formulations and devices for achieving sustained release are known in the art.

The binding agent (e.g., an antibody), binding agent-encoding polynucleotide, and/or vector containing such a polynucleotide may be administered to the patient in a carrier; preferably a pharmaceutically-acceptable carrier. Thus, in further aspects, the disclosure provides a composition (e.g., a pharmaceutical composition) comprising a pharmaceutically acceptable carrier and (a) a binding agent disclosed herein, (b) a binding agent-encoding polynucleotide disclosed herein and/or (c) a vector comprising a binding agent-encoding polynucleotide.

As used herein, “pharmaceutically acceptable carrier” or “pharmaceutically acceptable excipient” includes any material which, when combined with an active ingredient, allows the ingredient to retain biological activity and is non-reactive with the subject's immune system and non-toxic to the subject when delivered. Examples include, but are not limited to, any of the standard pharmaceutical carriers such as a phosphate buffered saline solution, water, emulsions such as oil/water emulsion, and various types of wetting agents. Non-limiting examples of diluents for aerosol or parenteral administration are phosphate buffered saline, normal (0.9%) saline, Ringer's solution and dextrose solution. The pH of the solution may be from about 5 to about 8, or from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

While any suitable carrier known to those of ordinary skill in the art may be employed in the pharmaceutical compositions of this disclosure, the type of carrier will vary depending on the mode of administration. Numerous delivery techniques for the pharmaceutical compositions of the disclosure (i.e., containing a binding agent or a binding agent-encoding polynucleotide) are well known in the art, such as those described by Rolland, 1998, Crit. Rev. Therap. Drug Carrier Systems 15:143-198, and references cited therein.

Compositions that include the binding agent and/or binding agent-encoding polynucleotide disclosed herein may be formulated for any appropriate manner of administration, including for example, systemic, topical, oral, nasal, intravenous, intracranial, intraperitoneal, subcutaneous or intramuscular administration, or by other methods, such as infusion, which ensure its delivery to the bloodstream in an effective form. The compositions may also be administered by isolated perfusion techniques, such as isolated tissue perfusion, to exert local therapeutic effects. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. In some embodiments, for oral administration, the formulation of the compositions is resistant to decomposition in the digestive tract, for example, as microcapsules encapsulating the binding agent (or binding agent-encoding polynucleotide or vector comprising such a polynucleotide) within liposomes. Biodegradable microspheres (e.g., polylactate polyglycolate) may also be employed as carriers for the pharmaceutical compositions of this disclosure. Suitable biodegradable microspheres are disclosed, for example, in U.S. Pat. Nos. 4,897,268 and 5,075,109.

The compositions may also comprise buffers (e.g., neutral buffered saline or phosphate buffered saline), carbohydrates (e.g., glucose, mannose, sucrose or dextran), mannitol, proteins, polypeptides or amino acids such as glycine, antioxidants, chelating agents such as EDTA or glutathione, adjuvants (e.g., aluminum hydroxide) and/or preservatives. Alternatively, the compositions may be formulated as a lyophilizate (e.g., for reconstitution prior to administration).

In some embodiments, the binding agent and/or binding agent-encoding polynucleotide also may be entrapped in microcapsules prepared, for example, by coacervation techniques or by interfacial polymerization (for example, hydroxymethylcellulose or gelatin-microcapsules and poly(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000. To increase the serum half life of the binding agent (e.g., an antibody), one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, and IgG4) that is responsible for increasing the in vivo serum half-life of the IgG molecule.

The binding agents (and/or binding agent-encoding polynucleotides) disclosed herein may also be formulated as liposomes. Liposomes containing the binding agents (and/or binding agent-encoding polynucleotides) are prepared by methods known in the art, such as described in Epstein et al., 1985, Proc. Natl. Acad. Sci. USA 82:3688; Hwang et al., 1980, Proc. Natl. Acad. Sci. USA 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. In addition, where the binding agent is an antibody, antibodies (including antigen binding domain fragments such as Fab′ fragments) can be conjugated to the liposomes as described in Martin et al., 1982, J. Biol. Chem. 257:286-288, via a disulfide interchange reaction. Administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding a polypeptide or antibody disclosed herein can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. The therapeutic polynucleotides and polypeptides of the present disclosure can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0 524 968. Additional approaches are described in Philip, Mol. Cell. Biol. (1994) 14:2411, and in Woffendin, Proc. Natl. Acad. Sci. (1994) 91:1581.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated.

The compositions of the disclosure include bulk drug compositions useful in the manufacture of non-pharmaceutical compositions (e.g., impure or non-sterile compositions) and pharmaceutical compositions (i.e., compositions that are suitable for administration to a subject or patient) that can be used in the preparation of unit dosage forms.

The compositions and methods disclosed herein can be used for treatment of patients having or at risk for hCMV infection, e.g., pregnant women, neonates, immunocompromised individuals, and individuals undergoing organ transplantation (e.g., hCMV negative individuals receiving organs (e.g., kidney, lung, liver, pancreas, or heart) from hCMV donors).

The compositions and methods disclosed herein can be used for treatment of hCMV-related disease, e.g., cytomegalic inclusion disease, cerebral calcification, cytomegalovirus hepatitis, cytomegalovirus retinitis, cytomegalovirus colitis, cytomegalovirus pneumonitis, cytomegalovirus esophagitis, polyradiculopathy, transverse myelitis, subacute encephalitis, cytomegalovirus mononucleosis, or arterial hypertension.

The following examples are provided to illustrate, but not to limit, the invention.

### EXAMPLES

### Example 1

### Isolation and Characterization of Anti-CMV Antibodies

Antibodies reactive against antigenic domain 4 (AD4) (amino acids 121-132 and 344-438) (Potzsch, 2011, PLoS Pathog., 7:e1002172) of CMV AD169 envelope glycoprotein B (gB) (SEQ ID NO: 1) were isolated from the plasma of a healthy volunteer having CMV-neutralizing activity as determined using an in vitro microneutralization assay. Heavy and light chain sequences of the antibodies were determined, resulting in the identification of 10 gamma, 4 kappa, and 10 lambda chains. The heavy and light chains were combinatorially paired and transiently expressed in HEK293 cells (Cheung et al., 2012, Nat. Biotechnol., doi:10.1038/nbt.2167). Fifteen monoclonal antibodies (as a result of pairing 5 gamma chains (SEQ ID NOs: 3-7) with 10 lambda (SEQ ID NOs: 8-17) and 3 kappa (SEQ ID NOs: 18-20) chains) showed significant AD4-binding specificity by ELISA titration and were further characterized for affinity measurement by Biacore T200 and tested for in vitro neutralization activity of CMV.

For affinity measurement, a CM5 chip was coated first with anti-human IgG capture antibody using amine coupling, then the antibodies were captured as the ligand, and serially diluted AD4 polypeptide (SEQ ID NO: 2) was used as the analyte. The kinetics analysis was done with the Biacore kinetics analysis software where the fitted curves for binding of the ligand to analyte were modeled as a 1:1 interaction. The antibodies possessed affinities ranging from 278 μM to 7.76 nM (Table 1).

For measurement of in vitro neutralization activity, 1×10⁶ MRCS fibroblast cells were seeded in clear bottom, black plasma coated 96-well plates one day prior to infection. On the day of infection, CMV strain AD169 (ATCC) at a titer that generates 100 to 200 infected cells/well was incubated with serial dilutions of antibody, plasma or buffer alone in 96-well plates at 37° C. for one hour. The virus-antibody mixture was then added to MRCS cells in the 96-well plates as described above and incubated at 37° C. with 5% CO₂. Infection was detected 16-20 hours later as follows. Medium was removed, and the cells were fixed with 100% ethanol for 30 minutes. The cells were rehydrated with 1×PBS for 30 minutes, then blocked with 5% normal goat serum in 1×PBS with 0.3% TRITON X100 for one hour. Anti-CMV immediate early (1E) antigen-specific mouse antibody (Light Diagnostics, Temecula, Calif., cat. no. MAB810) was diluted 10.000-fold in 1% BSA in 1×PBS with 0.3% TRITON X100 and added to the blocked wells and incubated for one hour at 37° C. The wells were washed, then ALEXA FLUOR 488-conjugated anti-mouse antibody (Cell Signaling Technology, cat. no. 4408) was added to visualize the nuclei of infected cells. Plates were scanned on the ACUMEN Explorer microplate cytometer (TTP Labtech), and the number of IE antigen-positive nuclei was quantified for each well. The number of positive nuclei in the wells where no virus was added (cells only) was considered background level and was subtracted from all other wells. The wells where no antibody was added (buffer only) to the virus were considered as 100% infection, and the infection levels of all other samples were calculated as the level of infected cells in terms of a percentage relative to the buffer only wells. Seven antibodies exhibited neutralizing activity with IC₅₀ values ranging from 0.04 μg/ml to 25 μg/ml (Table 1).

TABLE 1
Characteristics of anti-CMV antibodies

| | | | | | | CMV |
|---|---|---|---|---|---|---|
| Antibody | VH | VL | Kₒₙ (M⁻¹s⁻¹) | K_{off} (s⁻¹) | K_{D} (M) | Neutr. IC₅₀ |
| C1 | SEQ ID NO: 22 | SEQ ID NO: 67 | 4.67 × 10⁵ | 2.40 × 10⁻³ | 5.14 × 10⁻⁹ | — |
| C2 | SEQ ID NO: 22 | SEQ ID NO: 76 | 5.05 × 10⁵ | 2.11 × 10⁻³ | 4.17 × 10⁻⁹ | — |
| C3 | SEQ ID NO: 22 | SEQ ID NO: 85 | 1.24 × 10⁶ | 6.80 × 10⁻⁴ | 5.49 × 10⁻¹⁰ | <0.07 μg/ml |
| C4 | SEQ ID NO: 22 | SEQ ID NO: 94 | 9.08 × 10⁵ | 6.30 × 10⁻⁴ | 6.95 × 10⁻¹⁰ | <0.04 μg/ml |
| C5 | SEQ ID NO: 22 | SEQ ID NO: 103 | 7.21 × 10⁵ | 2.01 × 10⁻⁴ | 2.78 × 10⁻¹⁰ | <6 μg/ml |
| C6 | SEQ ID NO: 22 | SEQ ID NO: 112 | 5.54 × 10⁵ | 1.38 × 10⁻³ | 2.50 × 10⁻⁹ | <2 μg/ml |
| C7 | SEQ ID NO: 22 | SEQ ID NO: 121 | 7.79 × 10⁵ | 6.04 × 10⁻³ | 7.76 × 10⁻⁹ | — |
| C8 | SEQ ID NO: 22 | SEQ ID NO: 130 | 4.14 × 10⁵ | 1.86 × 10⁻³ | 4.49 × 10⁻⁹ | — |
| C9 | SEQ ID NO: 22 | SEQ ID NO: 139 | 6.02 × 10⁵ | 2.38 × 10⁻³ | 3.95 × 10⁻⁹ | — |
| C10 | SEQ ID NO: 22 | SEQ ID NO: 148 | 4.96 × 10⁵ | 1.97 × 10⁻³ | 3.97 × 10⁻⁹ | — |
| C11 | SEQ ID NO: 31 | SEQ ID NO: 139 | 1.92 × 10⁵ | 1.09 × 10⁻³ | 5.67 × 10⁻⁹ | — |
| C12 | SEQ ID NO: 40 | SEQ ID NO: 103 | 1.13 × 10⁶ | 3.69 × 10⁻³ | 3.27 × 10⁻⁹ | — |
| C13 | SEQ ID NO: 49 | SEQ ID NO: 157 | 4.05 × 10⁵ | 7.95 × 10⁻⁴ | 1.96 × 10⁻⁹ | <25 μg/ml |
| C14 | SEQ ID NO: 49 | SEQ ID NO: 166 | 1.96 × 10⁵ | 4.71 × 10⁻⁴ | 2.40 × 10⁻⁹ | <3 μg/ml |
| C15 | SEQ ID NO: 58 | SEQ ID NO: 175 | 3.63 × 10⁵ | 2.34 × 10⁻³ | 6.44 × 10⁻⁹ | <0.3 μg/ml |

—, neutralizing activity was not detected at the highest IgG concentration tested (50 μg/ml)

This example demonstrates the isolation and production of high affinity human antibodies with neutralizing activity against CMV.

### Example 2

### Antibody and Other Sequences

For each antibody chain, the full-length antibody chain (HC or LC), variable region nucleic acid sequence (Nuc), variable region (VH or VL), framework regions (FR1-FR4), and complementarity determining regions (CDR1-CDR3) are provided.

CMV AD169 gB
(SEQ ID NO: 1)
>gi|138192|sp|P06473.1|GB_HCMVA RecName: Full = Envelope glycoprotein B;
Short = gB; Flags: Precursor
MESRIWCLVVCVNLCIVCLGAAVSSSSTSHATSSTHNGSHTSRTTSAQTRSVYSQHVTSSEAVSHRANET
IYNTTLKYGDVVGVNTTKYPYRVCSMAQGTDLIRFERNIICTSMKPINEDLDEGIMVVYKRNIVAHTFKV
RVYQKVLTFRRSYAYIYTTYLLGSNTEYVAPPMWEIHHINKFAQCYSSYSRVIGGTVFVAYHRDSYENKT
MQLIPDDYSNTHSTRYVTVKDQWHSRGSTWLYRETCNLNCMLTITTARSKYPYHFFATSTGDVVYISPEY
NGTNRNASYFGENADKFFIFPNYTIVSDFGRPNAAPETHRLVAFLERADSVISWDIQDEKNVTCQLTFWE
ASERTIRSEAEDSYHFSSAKMTATFLSKKQEVNMSDSALDCVRDEAINKLQQTENTSYNQTYEKYGNVSV
FETSGGLVVFWQGIKQKSLVELERLANRSSLNITHRTRRSTSDNNTTHLSSMESVHNLVYAQLQFTYDTL
RGYINRALAQIAEAWCVDQRRTLEVEKELSKINPSAILSAIYNKPIAARFMGDVLGLASCVTINQTSVKV
LRDMNVKESPGRCYSRPVVIFNFANSSYVQYGQLGEDNEILLGNHRTEECQLPSLKIFIAGNSAYEYVDY
LFKRMIDLSSISTVDSMIALDIDPLENTDFRVLELYSQKELRSSNVFDLEEIMREFNSYKQRVKYVEDKV
VDPLPPYLKGLDDLMSGLGAAGKAVGVAIGAVGGAVASVVEGVATFLKNPFGAFTIILVAIAVVIITYLI
YTRQRRLCTQPLQNLFPYLVSADGTTVTSGSTKDTSLQAPPSYEESVYNSGRKGPGPPSSDASTAAPPYT
NEQAYQMLLALARLDAEQRAQQNGTDSLDGQTGTQDKGQKPNLLDRLRHRKNGYRHLKDSDEEENV
cloned AD4 fragment
(SEQ ID NO: 2)
TSMKPINEDLDEGIMVVYKRNIAGSGCQLTFWEASERTIRSEAEDSYHFSSAKMTATFLSKKQEVNMSDSALDC
VRDEAINKLQQIFNTSYNQTYEKYGNVSVFETSGGLVVFWQGIKQKS

### Heavy Chain 1

| | SEQ ID | |
|---|---|---|
| Region | NO | Sequence |
| HC | 3 | QVQLQESGPGLVKPLETLSLTCTVSGGSISSGSFYWTWIRQHPGKGLEW |
| | | IGYIHSSGTTYYNPSLKSRLNISRDTSKNQFTLKLSSVTAADTAVYYCA |
| | | KEIIQWPRRWFDPWGQGTLVTVSSASTKGPSVRPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHY |
| | | TQKSLSLSPGK |
| Nuc | 21 | CAGGTGCAGCTGCAGGAGTCGGGCCCAGGACTGGTGAAGCCTTTAGAGA |
| | | CCCTGTCCCTCACCTGCACTGTCTCTGGTGGCTCCATCAGCAGTGGTAG |
| | | TTTCTACTGGACCTGGATCCGCCAACACCCAGGGAAGGGCCTGGAGTGG |
| | | ATTGGGTACATCCATTCCAGTGGCACCACCTACTACAACCCGTCCCTCA |
| | | AGAGTCGACTTAACATATCAAGAGACACGTCTAAGAACCAGTTCACCCT |
| | | GAAACTGAGCTCGGTGACTGCCGCGGACACGGCCGTCTATTACTGTGCG |
| | | AAAGAGATTATACAATGGCCCCGGCGGTGGTTCGACCCCTGGGGCCAGG |
| | | GAACCCTGGTCACCGTCTCCTCA |
| VH | 22 | QVQLQESGPGLVKPLETLSLTCTVSGGSISSGSFYWTWIRQHPGKGLEW |
| | | IGYIHSSGTTYYNPSLKSRLNISRDTSKNQFTLKLSSVTAADTAVYYCA |
| | | KEIIQWPRRWFDPWGQGTLVTVSS |
| FR1 | 23 | QVQLQESGPGLVKPLETLSLTCTVSGGSIS |
| CDR1 | 24 | SGSFYWT |
| FR2 | 25 | WIRQHPGKGLEWIG |
| CDR2 | 26 | YIHSSGTTYYNPSLKS |
| FR3 | 27 | RLNISRDTSKNQFTLKLSSVTAADTAVYYCAK |
| CDR3 | 28 | EIIQWPRRWFDP |
| FR4 | 29 | WGQGTLVTVSS |

### Heavy Chain 2

| | SEQ ID | |
|---|---|---|
| Region | NO | Sequence |
| HC | 4 | EVQLLESGGGLIQPGGSLRLSCVASGFTFTRHAINWVRQAPGKGLEWVS |
| | | AISGSGSSTYYADSVKGRFTISRDNAKNTLYLQMNRLRVEDTAVYYCAK |
| | | DKDYGMVAATPDAFDIWGQGTMVTVSSASTKGPSVRPLAPSSKSTSGGT |
| | | AALGCLVKDYFPEPVTVSWNSGALTSGVHTFPAVLQSSGLYSLSSVVTV |
| | | PSSSLGTQTYICNVNHKPSNTKVDKRVEPKSCDKTHTCPPCPAPELLGG |
| | | PSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEALH |
| | | NHYTQKSLSLSPGK |
| Nuc | 30 | GAGGTGCAGCTGTTGGAGTCTGGGGGAGGCTTGATACAGCCTGGGGGGT |
| | | CCCTGAGACTCTCCTGTGTAGCCTCTGGATTCACCTTTACTAGACATGC |
| | | CATAAATTGGGTCCGCCAGGCTCCAGGGAAGGGGCTGGAGTGGGTCTCA |
| | | GCTATTAGTGGTAGTGGTAGTAGCACATATTACGCAGACTCCGTGAAGG |
| | | GCCGATTCACCATCTCCAGAGACAATGCCAAGAATACGCTGTATCTGCA |
| | | AATGAACAGACTGCGAGTCGAGGACACGGCCGTGTATTACTGTGCGAAA |
| | | GATAAGGATTACGGGATGGTAGCCGCTACACCAGATGCTTTTGATATCT |
| | | GGGGCCAAGGGACAATGGTCACCGTCTCTTCA |
| VH | 31 | EVQLLESGGGLIQPGGSLRLSCVASGFTFTRHAINWVRQAPGKGLEWVS |
| | | AISGSGSSTYYADSVKGRFTISRDNAKNTLYLQMNRLRVEDTAVYYCAK |
| | | DKDYGMVAATPDAFDIWGQGTMVTVSS |
| FR1 | 32 | EVQLLESGGGLIQPGGSLRLSCVASGFTFT |
| CDR1 | 33 | RHAIN |
| FR2 | 34 | WVRQAPGKGLEWVS |
| CDR2 | 35 | AISGSGSSTYYADSVKG |
| FR3 | 36 | RFTISRDNAKNTLYLQMNRLRVEDTAVYYCAK |
| CDR3 | 37 | DKDYGMVAATPDAFDI |
| FR4 | 38 | WGQGTMVTVSS |

### Heavy Chain 3

| | SEQ ID | |
|---|---|---|
| Region | NO | Sequence |
| HC | 5 | QVQLQESGPGLVKPLETLSLTCTVSGGSIRGGDYWWTWVRQPPGKGLEW |
| | | LGYIYRTGSTYYNPSLKSRLTISLDTSKNHFSLKLASATAADTAVYYCA |
| | | RDPGDDSGGNSGLDYWGQGVLVSVSSASTKGPSVRPLAPSSKSTSGGTA |
| | | ALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHN |
| | | HYTQKSLSLSPGK |
| Nuc | 39 | CAGGTGCAGTTGCAGGAGTCGGGCCCAGGACTGGTGAAGCCTTTAGAGA |
| | | CCCTGTCCCTCACCTGCACTGTGTCTGGTGGCTCCATCAGAGGTGGTGA |
| | | TTACTGGTGGACTTGGGTCCGCCAGCCCCCAGGTAAGGGCCTGGAGTGG |
| | | CTTGGCTACATATATCGCACTGGGAGCACCTACTACAATCCGTCCCTCA |
| | | AGAGTCGACTTACGATCTCATTGGACACGTCCAAGAACCACTTCTCCCT |
| | | GAAGCTGGCCTCGGCTACTGCCGCAGACACGGCCGTCTATTACTGTGCC |
| | | AGAGACCCAGGTGATGACTCCGGTGGAAACTCGGGCTTGGACTACTGGG |
| | | GCCAGGGAGTCCTCGTCTCCGTCTCCTCA |
| VH | 40 | QVQLQESGPGLVKPLETLSLTCTVSGGSIRGGDYWWTWVRQPPGKGLEW |
| | | LGYIYRTGSTYYNPSLKSRLTISLDTSKNHFSLKLASATAADTAVYYCA |
| | | RDPGDDSGGNSGLDYWGQGVLVSVSS |
| FR1 | 41 | QVQLQESGPGLVKPLETLSLTCTVSGGSIR |
| CDR1 | 42 | GGDYWWT |
| FR2 | 43 | WVRQPPGKGLEWLG |
| CDR2 | 44 | YIYRTGSTYYNPSLKS |
| FR3 | 45 | RLTISLDTSKNHFSLKLASATAADTAVYYCAR |
| CDR3 | 46 | DPGDDSGGNSGLDY |
| FR4 | 47 | WGQGVLVSVSS |

### Heavy Chain 4

| | SEQ | |
|---|---|---|
| | ID | |
| Region | NO | Sequence |
| HC | 6 | QVQLVESGGGLVKPGGSLRLSCTASGLTFSDYYLSWIRQAPGKGLEWIS |
| | | YISGFNTYTNYADSVRGRFTISRDNAKKSLYLQMDSLRVEDTAVYYCTG |
| | | ARRGESGAYGSSDYWGLGTLVTVSSASTKGPSVRPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKRVEPKSCDKTHTCPPCPAPELLGGPS |
| | | VFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTIS |
| | | KAKGQPREPQVYTLPPSREEMTKNQVSLTCLVKGFYPSDIAVEWESNGQ |
| | | PENNYKTTPPVLDSDGSFFLYSKLTVDKSRWQQGNVFSCSVMHEALHNH |
| | | YTQKSLSLSPGK |
| Nuc | 48 | CAGGTGCAGCTGGTGGAGTCTGGGGGAGGCTTGGTCAAGCCTGGAGGGT |
| | | CCCTGAGACTCTCCTGTACAGCCTCTGGATTGACCTTCAGTGACTACTA |
| | | CCTGAGCTGGATCCGCCAGGCTCCAGGGAAGGGGCTGGAGTGGATTTCA |
| | | TACATTAGTGGTTTCAATACTTACACAAACTATGCAGACTCTGTGAGGG |
| | | GCCGATTCACCATCTCCAGAGACAACGCCAAGAAATCACTGTATCTGCA |
| | | AATGGACAGCCTGAGAGTCGAGGACACGGCTGTCTATTATTGTACGGGG |
| | | GCCCGCAGGGGCGAGAGTGGGGCCTACGGGTCGAGTGACTACTGGGGCC |
| | | TGGGAACCCTGGTCACCGTCTCCTCA |
| VH | 49 | QVQLVESGGGLVKPGGSLRLSCTASGLTFSDYYLSWIRQAPGKGLEWIS |
| | | YISGFNTYTNYADSVRGRFTISRDNAKKSLYLQMDSLRVEDTAVYYCTG |
| | | ARRGESGAYGSSDYWGLGTLVTVSS |
| FR1 | 50 | QVQLVESGGGLVKPGGSLRLSCTASGLTFS |
| CDR1 | 51 | DYYLS |
| FR2 | 52 | WIRQAPGKGLEWIS |
| CDR2 | 53 | YISGFNTYTNYADSVRG |
| FR3 | 54 | RFTISRDNAKKSLYLQMDSLRVEDTAVYYCTG |
| CDR3 | 55 | ARRGESGAYGSSDY |
| FR4 | 56 | WGLGTLVTVSS |

### Heavy Chain 5

| | SEQ | |
|---|---|---|
| | ID | |
| Region | NO | Sequence |
| HC | 7 | EMQLLESGGGLVQPGGSLRVSCAASGFTFSSHAMSWVRQAPGKGLEWVS |
| | | SISRSGDNTFYADSVKGRFTISRDNIKSTVYLQMNSLRAEDTAVYFCAS |
| | | RYTEGDSVWYFDVWGRGTLVTVSSASTKGPSVRPLAPSSKSTSGGTAAL |
| | | GCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISK |
| | | AKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHY |
| | | TQKSLSLSPGK |
| Nuc | 57 | GAGATGCAGCTGTTGGAGTCTGGGGGAGGCTTGGTACAGCCTGGGGGGT |
| | | CCCTGAGAGTCTCCTGTGCAGCCTCTGGATTCACCTTTAGCAGTCATGC |
| | | CATGAGTTGGGTCCGCCAGGCTCCAGGGAAGGGGCTGGAGTGGGTCTCA |
| | | AGTATAAGTCGAAGTGGTGATAACACATTCTACGCAGACTCCGTGAAGG |
| | | GCCGCTTCACCATCTCCAGAGACAACATTAAGAGTACAGTGTATCTGCA |
| | | AATGAACAGCCTGAGAGCCGAGGACACGGCCGTATATTTCTGTGCGAGT |
| | | CGATATACGGAAGGTGACTCCGTCTGGTACTTCGATGTCTGGGGCCGGG |
| | | GCACCCTGGTCACTGTCTCCTCA |
| VH | 58 | EMQLLESGGGLVQPGGSLRVSCAASGFTFSSHAMSWVRQAPGKGLEWVS |
| | | SISRSGDNTFYADSVKGRFTISRDNIKSTVYLQMNSLRAEDTAVYFCAS |
| | | RYTEGDSVWYFDVWGRGTLVTVSS |
| FR1 | 59 | EMQLLESGGGLVQPGGSLRVSCAASGFTFS |
| CDR1 | 60 | SHAMS |
| FR2 | 61 | WVRQAPGKGLEWVS |
| CDR2 | 62 | SISRSGDNTFYADSVKG |
| FR3 | 63 | RFTISRDNIKSTVYLQMNSLRAEDTAVYFCAS |
| CDR3 | 64 | RYTEGDSVWYFDV |
| FR4 | 65 | WGRGTLVTVSS |

### Light Chain 1

| | SEQ | |
|---|---|---|
| | ID | |
| Region | NO | Sequence |
| LC | 8 | LFVLTQPPSASGTAGQRVTISCSASNSNIGTNTVTWYQHLPGAAPRLLI |
| | | YNNDQRPSGVPDRFSGSRSGTSASLAISGLQSEDETVYYCSAWDDSLNG |
| | | VVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 66 | CTGTTTGTGTTGACTCAGCCGCCCTCAGCGTCAGGGACCGCCGGGCAGA |
| | | GGGTCACCATCTCTTGTTCTGCAAGCAACTCCAACATCGGAACTAATAC |
| | | TGTAACCTGGTACCAGCATCTCCCAGGAGCGGCCCCCAGACTCCTCATC |
| | | TATAATAATGATCAGAGGCCCTCAGGGGTCCCTGACCGATTCTCTGGCT |
| | | CCAGGTCTGGCACCTCAGCCTCCCTGGCCATCAGTGGGCTCCAGTCTGA |
| | | GGATGAGACTGTTTATTACTGTTCAGCATGGGATGACAGCCTGAATGGC |
| | | GTGGTGTTCGGCGGAGGGACCAAACTGACCGTCCTAGGTCAGCCC |
| VL | 67 | LFVLTQPPSASGTAGQRVTISCSASNSNIGTNTVTWYQHLPGAAPRLLI |
| | | YNNDQRPSGVPDRFSGSRSGTSASLAISGLQSEDETVYYCSAWDDSLNG |
| | | VVFGGGTKLTVL |
| FR1 | 68 | LFVLTQPPSASGTAGQRVTISC |
| CDR1 | 69 | SASNSNIGTNTVT |
| FR2 | 70 | WYQHLPGAAPRLLIY |
| CDR2 | 71 | NNDQRPS |
| FR3 | 72 | GVPDRFSGSRSGTSASLAISGLQSEDETVYYC |
| CDR3 | 73 | AWDDSLNGVV |
| FR4 | 74 | FGGGTKLTVL |

### Light Chain 2

| | SEQ | |
|---|---|---|
| | ID | |
| Region | NO | Sequence |
| LC | 9 | LFVLTQPPSASGTPGQRVTISCSASSSSIGTNTVTWYQHLPGAAPKLLI |
| | | YNNDQRPSGIPDRFSGSKSGTSASLAITGLQSEDETVYYCAAWDDSLNG |
| | | VVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 75 | CTCTTTGTGTTGACTCAGCCACCCTCAGCGTCAGGGACCCCCGGGCAGA |
| | | GGGTCACCATCTCTTGTTCTGCAAGCAGCTCCAGCATCGGAACTAACAC |
| | | CGTAACCTGGTACCAGCATCTCCCAGGAGCGGCCCCCAAACTCCTAATC |
| | | TATAATAATGATCAGCGGCCCTCAGGGATCCCTGACCGATTTTCTGGCT |
| | | CCAAGTCTGGCACCTCAGCCTCCCTGGCCATCACTGGGCTCCAGTCTGA |
| | | GGATGAGACTGTTTATTACTGTGCAGCATGGGATGACAGTCTGAATGGC |
| | | GTGGTTTTCGGCGGAGGGACCAAGCTGACCGTCCTAGGTCAGCCC |
| VL | 76 | LFVLTQPPSASGTPGQRVTISCSASSSSIGTNTVTWYQHLPGAAPKLLI |
| | | YNNDQRPSGIPDRFSGSKSGTSASLAITGLQSEDETVYYCAAWDDSLNG |
| | | VVFGGGTKLTVL |
| FR1 | 77 | LFVLTQPPSASGTPGQRVTISC |
| CDR1 | 78 | SASSSSIGTNTVT |
| FR2 | 79 | WYQHLPGAAPKLLIY |
| CDR2 | 80 | NNDQRPS |
| FR3 | 81 | GIPDRFSGSKSGTSASLAITGLQSEDETVYYC |
| CDR3 | 82 | AAWDDSLNGVV |
| FR4 | 83 | FGGGTKLTVL |

### Light Chain 3

| | SEQ | |
|---|---|---|
| | ID | |
| Region | NO | Sequence |
| LC | 10 | QSMLTQPPSVSAAPGQKVIISCSGSSSNIGNNYVNWYQQLPGTAPKLLI |
| | | YDNDKRPSGIPDRFSGSKSGTSATLGITGLQTGDEADYYCGTWDSTLTA |
| | | GVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 84 | CAGTCTATGTTGACGCAGCCGCCCTCAGTGTCTGCGGCCCCAGGACAGA |
| | | AGGTCATCATCTCCTGCTCTGGAAGCAGCTCCAACATTGGGAATAACTA |
| | | TGTGAACTGGTACCAGCAGCTCCCAGGAACAGCCCCCAAACTCCTCATT |
| | | TATGACAATGATAAGCGACCCTCAGGGATTCCTGACCGATTCTCTGGCT |
| | | CCAAGTCTGGCACGTCGGCCACCCTGGGCATCACCGGACTCCAGACTGG |
| | | GGACGAGGCCGATTATTACTGCGGAACATGGGATAGCACCCTGACTGCG |
| | | GGGGTGTTCGGCGGAGGGACCAAGTTGACCGTCCTGGGTCAGCCC |
| VL | 85 | QSMLTQPPSVSAAPGQKVIISCSGSSSNIGNNYVNWYQQLPGTAPKLLI |
| | | YDNDKRPSGIPDRFSGSKSGTSATLGITGLQTGDEADYYCGTWDSTLTA |
| | | GVFGGGTKLTVL |
| FR1 | 86 | QSMLTQPPSVSAAPGQKVIISC |
| CDR1 | 87 | SGSSSNIGNNYVN |
| FR2 | 88 | WYQQLPGTAPKLLIY |
| CDR2 | 89 | DNDKRPS |
| FR3 | 90 | GIPDRFSGSKSGTSATLGITGLQTGDEADYYC |
| CDR3 | 91 | GTWDSTLTAGV |
| FR4 | 92 | FGGGTKLTVL |

### Light Chain 4

| | SEQ | |
|---|---|---|
| | ID | |
| Region | NO | Sequence |
| LC | 11 | QSVLTQPPSVSAAPGQKVIISCSGSGSNIGSHYVNWYQQLPGAAPKLLI |
| | | YDNDKRPSGIPDRFSGSKSGTSATLAITGLQTGDEADYHCATWDGTLTA |
| | | GVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 93 | CAGTCTGTCTTGACACAGCCGCCCTCAGTGTCTGCGGCCCCTGGACAGA |
| | | AGGTCATCATTTCCTGCTCTGGCAGCGGCTCCAACATTGGCAGTCATTA |
| | | TGTAAATTGGTATCAGCAGCTCCCAGGAGCAGCCCCCAAACTCCTCATT |
| | | TATGACAATGATAAGCGACCCTCAGGGATTCCTGACCGATTCTCTGGCT |
| | | CCAAGTCTGGCACGTCAGCCACCCTGGCCATCACCGGACTCCAGACTGG |
| | | GGACGAGGCCGACTATCACTGCGCAACATGGGATGGCACCCTCACTGCG |
| | | GGGGTGTTCGGCGGAGGGACCAAGCTGACCGTCCTAGGTCAGCCC |
| VL | 94 | QSVLTQPPSVSAAPGQKVIISCSGSGSNIGSHYVNWYQQLPGAAPKLLI |
| | | YDNDKRPSGIPDRFSGSKSGTSATLAITGLQTGDEADYHCATWDGTLTA |
| | | GVFGGGTKLTVL |
| FR1 | 95 | QSVLTQPPSVSAAPGQKVIISC |
| CDR1 | 96 | SGSGSNIGSHYVN |
| FR2 | 97 | WYQQLPGAAPKLLIY |
| CDR2 | 98 | DNDKRPS |
| FR3 | 99 | GIPDRFSGSKSGTSATLAITGLQTGDEADYHC |
| CDR3 | 100 | ATWDGTLTAGV |
| FR4 | 101 | FGGGTKLTVL |

### Light Chain 5

| | SEQ | |
|---|---|---|
| | ID | |
| Region | NO | Sequence |
| LC | 12 | QSMLTQPPSVSAAPGQKVIISCSGSSSNIGNNFVNWYQKFPGTAPKLLI |
| | | YDNDKRPSGIPDRFSGSKSGTSATLGITGLQTGDEAEYYCGTWDSSLTA |
| | | GVFGGGTTLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 102 | CAGTCTATGTTGACGCAGCCGCCCTCAGTGTCTGCGGCCCCAGGACAGA |
| | | AGGTCATCATCTCCTGCTCTGGAAGCAGCTCCAACATTGGAAATAACTT |
| | | TGTAAACTGGTACCAGAAGTTCCCAGGAACAGCCCCCAAACTCCTCATT |
| | | TATGACAATGATAAGCGACCCTCAGGGATTCCTGACCGATTCTCTGGCT |
| | | CCAAGTCCGGCACGTCAGCCACCCTGGGCATCACCGGACTCCAGACTGG |
| | | GGACGAGGCCGAATATTATTGCGGAACATGGGATAGCAGCCTGACTGCG |
| | | GGGGTGTTCGGCGGAGGGACCACGCTGACCGTCCTGGGTCAGCCC |
| VL | 103 | QSMLTQPPSVSAAPGQKVIISCSGSSSNIGNNFVNWYQKFPGTAPKLLI |
| | | YDNDKRPSGIPDRFSGSKSGTSATLGITGLQTGDEAEYYCGTWDSSLTA |
| | | GVFGGGTTLTVL |
| FR1 | 104 | QSMLTQPPSVSAAPGQKVIISC |
| CDR1 | 105 | SGSSSNIGNNFVN |
| FR2 | 106 | WYQKFPGTAPKLLIY |
| CDR2 | 107 | DNDKRPS |
| FR3 | 108 | GIPDRFSGSKSGTSATLGITGLQTGDEAEYYC |
| CDR3 | 109 | GTWDSSLTAGV |
| FR4 | 110 | FGGGTTLTVL |

### Light Chain 6

| | SEQ | |
|---|---|---|
| | ID | |
| Region | NO | Sequence |
| LC | 13 | QSVLTQPPSASGTPGQRVTISCSGSSSNIGSNTVTWYQQLPGTAPKLLI |
| | | SRNNQRPSGVPDRFSGSKSGTSASLAISGLQSEDEADYYCSSWDDSLNG |
| | | VVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 111 | CAGTCTGTGCTGACTCAGCCACCCTCAGCGTCTGGGACCCCCGGGCAGA |
| | | GGGTCACCATCTCTTGTTCTGGAAGCAGCTCCAACATCGGAAGTAATAC |
| | | TGTAACCTGGTACCAGCAGCTCCCAGGAACGGCCCCCAAACTCCTCATC |
| | | TCTCGTAATAATCAGCGGCCCTCAGGGGTCCCTGACCGATTCTCTGGCT |
| | | CCAAGTCTGGCACCTCAGCCTCCCTGGCCATCAGTGGGCTCCAGTCTGA |
| | | GGATGAGGCTGATTATTATTGTTCATCATGGGATGACAGCCTGAATGGT |
| | | GTGGTGTTCGGCGGAGGGACCAAGCTGACCGTCCTAGGTCAGCCC |
| VL | 112 | QSVLTQPPSASGTPGQRVTISCSGSSSNIGSNTVTWYQQLPGTAPKLLI |
| | | SRNNQRPSGVPDRFSGSKSGTSASLAISGLQSEDEADYYCSSWDDSLNG |
| | | VVFGGGTKLTVL |
| FR1 | 113 | QSVLTQPPSASGTPGQRVTISC |
| CDR1 | 114 | SGSSSNIGSNTVT |
| FR2 | 115 | WYQQLPGTAPKLLIS |
| CDR2 | 116 | RNNQRPS |
| FR3 | 117 | GVPDRFSGSKSGTSASLAISGLQSEDEADYYC |
| CDR3 | 118 | SSWDDSLNGVV |
| FR4 | 119 | FGGGTKLTVL |

### Light Chain 7

| | SEQ | |
|---|---|---|
| | ID | |
| Region | NO | Sequence |
| LC | 14 | QSVLTQPPSASGTPGQRVTISCSGSSSNIGSNTVNWYQQVPGTAPRLLI |
| | | YSHNQRPSGVPDRFSGSKSGTSASLAISGLQSEDEADYYCASWDDSLNG |
| | | VVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 120 | CAGTCTGTGCTGACTCAGCCACCCTCAGCGTCTGGGACCCCCGGGCAGA |
| | | GGGTCACCATCTCTTGTTCTGGGAGCAGCTCCAACATCGGAAGTAATAC |
| | | TGTAAACTGGTACCAGCAGGTCCCAGGAACGGCCCCCAGACTCCTCATC |
| | | TATAGTCATAATCAGCGGCCCTCAGGGGTCCCTGACCGATTCTCTGGCT |
| | | CCAAGTCTGGCACCTCAGCCTCCCTGGCCATCAGTGGCCTCCAGTCTGA |
| | | GGATGAGGCTGATTATTACTGTGCATCATGGGATGACAGCCTGAATGGT |
| | | GTGGTATTCGGCGGAGGGACCAAGCTGACCGTCCTAGGTCAGCCC |
| VL | 121 | QSVLTQPPSASGTPGQRVTISCSGSSSNIGSNTVNWYQQVPGTAPRLLI |
| | | YSHNQRPSGVPDRFSGSKSGTSASLAISGLQSEDEADYYCASWDDSLNG |
| | | VVFGGGTKLTVL |
| FR1 | 122 | QSVLTQPPSASGTPGQRVTISC |
| CDR1 | 123 | SGSSSNIGSNTVN |
| FR2 | 124 | WYQQVPGTAPRLLIY |
| CDR2 | 125 | SHNQRPS |
| FR3 | 126 | GVPDRFSGSKSGTSASLAISGLQSEDEADYYC |
| CDR3 | 127 | ASWDDSLNGVV |
| FR4 | 128 | FGGGTKLTVL |

### Light Chain 8

| | SEQ | |
|---|---|---|
| | ID | |
| Region | NO | Sequence |
| LC | 15 | QSVLTQPPSVSAAPGQKVTISCSGSRSNIGKNYVYWYQQLPGTAPKLLI |
| | | YDNNKRPSGIPDRFSGSKSGTSATLAITGLQTGDEADYYCGTWDSSLKT |
| | | GIFFGGGTKLTVLGQPKAAPSVTLFPPSSEELQANKATLVCLISDFYPG |
| | | AVTVAWKADSSPVKAGVETTTPSKQSNNKYAASSYLSLTPEQWKSHKSY |
| | | SCQVTHEGSTVEKTVAPTECS |
| Nuc | 129 | CAGTCTGTGTTGACGCAGCCGCCCTCAGTGTCTGCGGCCCCGGGACAGA |
| | | AGGTCACCATCTCCTGCTCTGGAAGCAGATCCAACATTGGGAAGAATTA |
| | | TGTATACTGGTACCAGCAACTCCCAGGAACAGCCCCCAAACTCCTCATT |
| | | TATGACAATAATAAGCGACCCTCAGGGATTCCTGACCGATTCTCTGGCT |
| | | CCAAGTCTGGCACGTCGGCCACCCTGGCCATCACCGGCCTCCAGACTGG |
| | | GGACGAGGCCGATTATTACTGCGGAACATGGGATAGCAGCCTGAAAACT |
| | | GGCATTTTTTTCGGCGGAGGGACCAAGCTGACCGTCCTAGGTCAGCCC |
| VL | 130 | QSVLTQPPSVSAAPGQKVTISCSGSRSNIGKNYVYWYQQLPGTAPKLLI |
| | | YDNNKRPSGIPDRFSGSKSGTSATLAITGLQTGDEADYYCGTWDSSLKT |
| | | GIFFGGGTKLTVL |
| FR1 | 131 | QSVLTQPPSVSAAPGQKVTISC |
| CDR1 | 132 | SGSRSNIGKNYVY |
| FR2 | 133 | WYQQLPGTAPKLLIY |
| CDR2 | 134 | DNNKRPS |
| FR3 | 135 | GIPDRFSGSKSGTSATLAITGLQTGDEADYYC |
| CDR3 | 136 | GTWDSSLKTGIF |
| FR4 | 137 | FGGGTKLTVL |

### Light Chain 9

| | SEQ | |
|---|---|---|
| | ID | |
| Region | NO | Sequence |
| LC | 16 | QSVLTQPPSVSAAPGQKVTISCSGSRSNIAKNYVYWYQQLPGTAPKLLI |
| | | YDTNKRPSGIPDRFSGSKSGTSATLAITGLQTGDEADYYCGTWDSSLKT |
| | | AIFFGGGTTVTVLGQPKAAPSVTLFPPSSEELQANKATLVCLISDFYPG |
| | | AVTVAWKADSSPVKAGVETTTPSKQSNNKYAASSYLSLTPEQWKSHKSY |
| | | SCQVTHEGSTVEKTVAPTECS |
| Nuc | 138 | CAGTCTGTGTTGACGCAGCCGCCCTCAGTGTCTGCGGCCCCGGGACAGA |
| | | AGGTCACCATCTCCTGCTCTGGAAGCAGATCCAACATTGCGAAGAATTA |
| | | TGTATATTGGTATCAACAACTCCCAGGAACAGCCCCCAAACTCCTCATT |
| | | TATGACACTAATAAGCGACCCTCAGGGATTCCTGACAGATTCTCTGGCT |
| | | CCAAGTCTGGCACGTCGGCCACCCTGGCCATCACCGGCCTCCAGACTGG |
| | | GGACGAGGCCGATTATTACTGCGGAACATGGGATAGCAGCCTGAAAACT |
| | | GCCATTTTTTTCGGCGGAGGGACCACGGTGACCGTCCTAGGTCAGCCC |
| VL | 139 | QSVLTQPPSVSAAPGQKVTISCSGSRSNIAKNYVYWYQQLPGTAPKLLI |
| | | YDTNKRPSGIPDRFSGSKSGTSATLAITGLQTGDEADYYCGTWDSSLKT |
| | | AIFFGGGTTVTVL |
| FR1 | 140 | QSVLTQPPSVSAAPGQKVTISC |
| CDR1 | 141 | SGSRSNIAKNYVY |
| FR2 | 142 | WYQQLPGTAPKLLIY |
| CDR2 | 143 | DTNKRPS |
| FR3 | 144 | GIPDRFSGSKSGTSATLAITGLQTGDEADYYC |
| CDR3 | 145 | GTWDSSLKTAIF |
| FR4 | 146 | FGGGTTVTVL |

### Light Chain 10

| | SEQ | |
|---|---|---|
| | ID | |
| Region | NO | Sequence |
| LC | 17 | QSVLTQPPSASGTPGQRVTISCSGSSSNIGSNYIDWYQQLPGTGPQLLT |
| | | YRNNQRPSGVPDRFSGSKSGTSASLAISGLRSEDEADYYCASWDDSLSS |
| | | PVFGGGTKLTVLGQPKAAPSVTLFPPSSEELQANKATLVCLISDFYPGA |
| | | VTVAWKADSSPVKAGVETTTPSKQSNNKYAASSYLSLTPEQWKSHKSYS |
| | | CQVTHEGSTVEKTVAPTECS |
| Nuc | 147 | CAGTCTGTACTGACTCAGCCACCCTCAGCGTCTGGGACCCCCGGGCAGA |
| | | GGGTCACCATCTCTTGTTCTGGAAGCAGCTCCAACATCGGAAGTAATTA |
| | | TATAGACTGGTACCAGCAGCTCCCAGGAACTGGCCCCCAACTCCTCACT |
| | | TATAGGAACAATCAGCGGCCCTCAGGGGTCCCTGACCGCTTCTCTGGCT |
| | | CCAAGTCTGGCACCTCAGCCTCCCTGGCCATCAGTGGGCTCCGGTCCGA |
| | | GGATGAGGCTGATTATTACTGTGCATCATGGGATGACAGTCTGAGTAGT |
| | | CCCGTATTCGGCGGAGGGACCAAGCTGACCGTCCTAGGTCAGCCC |
| VL | 148 | QSVLTQPPSASGTPGQRVTISCSGSSSNIGSNYIDWYQQLPGTGPQLLT |
| | | YRNNQRPSGVPDRFSGSKSGTSASLAISGLRSEDEADYYCASWDDSLSS |
| | | PVFGGGTKLTVL |
| FR1 | 149 | QSVLTQPPSASGTPGQRVTISC |
| CDR1 | 150 | SGSSSNIGSNYID |
| FR2 | 151 | WYQQLPGTGPQLLTY |
| CDR2 | 152 | RNNQRPS |
| FR3 | 153 | GVPDRFSGSKSGTSASLAISGLRSEDEADYYC |
| CDR3 | 154 | ASWDDSLSSPV |
| FR4 | 155 | FGGGTKLTVL |

### Light Chain 11

| | SEQ | |
|---|---|---|
| | ID | |
| Region | NO | Sequence |
| LC | 18 | AIRMTQSPSSFSASTGDRVTITCRASQGISNYLAWYQQKPGKAPKLLIY |
| | | AASTLQSGVPSRFSGSGSGTDFTLTISCLQSEDFATYYCQQYYSYPFTF |
| | | GPGTKVDIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| Nuc | 156 | GCCATCCGGATGACCCAGTCTCCATCCTCATTCTCTGCATCTACAGGAG |
| | | ACAGAGTCACCATCACTTGTCGGGCGAGTCAGGGTATTAGCAATTATTT |
| | | AGCCTGGTATCAGCAAAAACCAGGGAAAGCCCCTAAGCTCCTGATCTAT |
| | | GCTGCATCCACTTTGCAAAGTGGGGTCCCATCAAGGTTCAGCGGCAGTG |
| | | GATCTGGGACAGATTTCACTCTCACCATCAGCTGCCTGCAGTCTGAAGA |
| | | TTTTGCAACTTATTACTGTCAACAGTATTATAGTTACCCATTCACTTTC |
| | | GGCCCTGGGACCAAAGTGGATATCAAACGA |
| VL | 157 | AIRMTQSPSSFSASTGDRVTITCRASQGISNYLAWYQQKPGKAPKLLIY |
| | | AASTLQSGVPSRFSGSGSGTDFTLTISCLQSEDFATYYCQQYYSYPFTF |
| | | GPGTKVDIK |
| FR1 | 158 | AIRMTQSPSSFSASTGDRVTITC |
| CDR1 | 159 | RASQGISNYLA |
| FR2 | 160 | WYQQKPGKAPKLLIY |
| CDR2 | 161 | AASTLQS |
| FR3 | 162 | GVPSRFSGSGSGTDFTLTISCLQSEDFATYYC |
| CDR3 | 163 | QQYYSYPFT |
| FR4 | 164 | FGPGTKVDIK |

### Light Chain 12

| | SEQ | |
|---|---|---|
| | ID | |
| Region | NO | Sequence |
| LC | 19 | AIRMTQSPSSFSASTGDRVTITCRASQGISNYLAWYQQKPGKAPKLLIY |
| | | AASTLQSGVPSRFSGSGSGTDFTLTISCLQSEDFATYYCQQYYSYPFTF |
| | | GPGTKVDIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| Nuc | 165 | GCCATCCGGATGACCCAGTCTCCATCCTCATTCTCTGCATCTACAGGAG |
| | | ACAGAGTCACCATCACTTGTCGGGCGAGTCAGGGTATTAGCAATTATTT |
| | | AGCCTGGTATCAGCAAAAACCAGGGAAAGCCCCTAAGCTCCTGATCTAT |
| | | GCTGCATCCACTTTGCAAAGTGGGGTCCCATCAAGGTTCAGCGGCAGTG |
| | | GATCTGGGACAGATTTCACTCTCACCATCAGCTGCCTGCAGTCTGAAGA |
| | | TTTTGCAACTTATTACTGTCAACAGTATTATAGTTACCCATTCACTTTC |
| | | GGCCCTGGGACCAAAGTGGATATCAAACGA |
| VL | 166 | AIRMTQSPSSFSASTGDRVTITCRASQGISNYLAWYQQKPGKAPKLLIY |
| | | AASTLQSGVPSRFSGSGSGTDFTLTISCLQSEDFATYYCQQYYSYPFTF |
| | | GPGTKVDIK |
| FR1 | 167 | AIRMTQSPSSFSASTGDRVTITC |
| CDR1 | 168 | RASQGISNYLA |
| FR2 | 169 | WYQQKPGKAPKLLIY |
| CDR2 | 170 | AASTLQS |
| FR3 | 171 | GVPSRFSGSGSGTDFTLTISCLQSEDFATYYC |
| CDR3 | 172 | QQYYSYPFT |
| FR4 | 173 | FGPGTKVDIK |

### Light Chain 13

| | SEQ | |
|---|---|---|
| | ID | |
| Region | NO | Sequence |
| LC | 20 | EVVLTQSPATLSVSAGDRATLSCRASQSVSRDLAWYQQKPGQAPRLLIY |
| | | GASTRATDIPVRFSGSGSGTEFSLTISSLQSEDFAVYYCHQYKHWPRTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACEV |
| | | THQGLSSPVTKSFNRGEC |
| Nuc | 174 | GAAGTAGTGCTGACTCAATCTCCAGCCACCCTGTCTGTGTCTGCAGGGG |
| | | ATAGAGCCACCCTCTCCTGCAGGGCCAGCCAGAGTGTTAGCCGCGACTT |
| | | AGCCTGGTATCAGCAGAAACCTGGCCAGGCTCCCAGGCTCCTCATCTAT |
| | | GGTGCTTCCACCAGGGCCACTGATATCCCAGTCAGGTTCAGTGGCAGTG |
| | | GGTCTGGGACAGAGTTCTCTCTCACCATCAGCAGCCTGCAGTCTGAAGA |
| | | TTTTGCAGTTTATTACTGTCATCAATATAAACACTGGCCTCGGACGTTC |
| | | GGCCAGGGGACCAAGGTGGAAATCAAGCGA |
| VL | 175 | EVVLTQSPATLSVSAGDRATLSCRASQSVSRDLAWYQQKPGQAPRLLIY |
| | | GASTRATDIPVRFSGSGSGTEFSLTISSLQSEDFAVYYCHQYKHWPRTF |
| | | GQGTKVEIK |
| FR1 | 176 | EVVLTQSPATLSVSAGDRATLSC |
| CDR1 | 177 | RASQSVSRDLA |
| FR2 | 178 | WYQQKPGQAPRLLIY |
| CDR2 | 179 | GASTRAT |
| FR3 | 180 | DIPVRFSGSGSGTEFSLTISSLQSEDFAVYYC |
| CDR3 | 181 | HQYKHWPRT |
| FR4 | 182 | FGQGTKVEIK |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A purified anti-human cytomegalovirus envelope glycoprotein B antibody comprising:
(i) (a) a heavy chain variable region having CDRs 1, 2 and 3 comprising the amino acid sequences set forth in SEQ ID NOs: 24, 26 and 28, respectively, and
(b) a light chain variable region having CDRs 1, 2 and 3 comprising a set of amino acid sequences set forth in
(i) SEQ ID NOs: 69, 71 and 73, respectively;
(ii) SEQ ID NOs: 78, 80 and 82, respectively;
(iii) SEQ ID NOs: 87, 89 and 91, respectively;
(iv) SEQ ID NOs: 96, 98 and 100, respectively;
(v) SEQ ID NOs: 105, 107 and 109, respectively;
(vi) SEQ ID NOs: 114, 116 and 118, respectively;
(vii) SEQ ID NOs: 123, 125 and 127, respectively;
(viii) SEQ ID NOs: 132, 134 and 136, respectively;
(ix) SEQ ID NOs: 141, 143 and 145, respectively; or
(x) SEQ ID NOs: 150, 152 and 154, respectively;
(ii) a heavy chain variable region having CDRs 1, 2 and 3 comprising the amino acid sequences set forth in SEQ ID NOs: 33, 35 and 37, respectively, and a light chain variable region having CDRs 1, 2 and 3 comprising the amino acid sequences set forth in SEQ ID NOs: 141, 143 and 145, respectively;
(iii) a heavy chain variable region having CDRs 1, 2 and 3 comprising the amino acid sequences set forth in SEQ ID NOs: 42, 44 and 46, respectively, and a light chain variable region having CDRs 1, 2 and 3 comprising the amino acid sequences set forth in SEQ ID NOs: 105, 107 and 109, respectively;
(iv) a heavy chain variable region having CDRs 1, 2 and 3 comprising the amino acid sequences set forth in SEQ ID NOs: 51, 53 and 55, respectively, and a light chain variable region having CDRs 1, 2 and 3 comprising the amino acid sequences set forth in SEQ ID NOs: 159, 161 and 163, respectively, or SEQ ID NOs: 168, 170 and 172, respectively; or
(v) a heavy chain variable region having CDRs 1, 2 and 3 comprising the amino acid sequences set forth in SEQ ID NOs: 60, 62 and 64, respectively, and a light chain variable region having CDRs 1, 2 and 3 comprising the amino acid sequences set forth in SEQ ID NOs: 177, 179 and 181, respectively.

2. A purified anti-human cytomegalovirus envelope glycoprotein B antibody comprising a heavy chain variable region comprising the sequence of SEQ ID NO: 22, 31, 40, 49, or 58 or a light chain variable region comprising the sequence of SEQ ID NO: 67, 76, 85, 94, 103, 112, 121, 130, 139, 148, 157, 166, or 175.

3. A purified anti-human cytomegalovirus envelope glycoprotein B antibody comprising:
(a) a heavy chain variable region comprising an amino acid sequence at least 80% identical to SEQ ID NO: 22 and a light chain variable region comprising an amino acid sequence at least 80% identical to SEQ ID NO: 67, 76, 85, 94, 103, 112, 121, 130, 139, or 148;
(b) a heavy chain variable region comprising an amino acid sequence at least 80% identical to SEQ ID NO: 31 and a light chain variable region comprising an amino acid sequence at least 80% identical to SEQ ID NO: 139;
(c) a heavy chain variable region comprising an amino acid sequence at least 80% identical to SEQ ID NO: 40 and a light chain variable region comprising an amino acid sequence at least 80% identical to SEQ ID NO: 103;
(d) a heavy chain variable region comprising an amino acid sequence at least 80% identical to SEQ ID NO: 49 and a light chain variable region comprising an amino acid sequence at least 80% identical to SEQ ID NO: 157 or 166; or
(e) a heavy chain variable region comprising an amino acid sequence at least 80% identical to SEQ ID NO: 58 and a light chain variable region comprising an amino acid sequence at least 80% identical to SEQ ID NO: 175.

4. A purified anti-human cytomegalovirus envelope glycoprotein B antibody, wherein the antibody:
(a) binds to a polypeptide consisting of SEQ ID NO: 2 with a dissociation constant of 1×10⁻⁸ M or less;
(b) binds to a polypeptide consisting of SEQ ID NO: 2 with an association rate of 1×10⁵ m⁻¹s⁻¹ or greater; or
(c) binds to a polypeptide consisting of SEQ ID NO: 2 with a dissociation rate of 1×10⁻² s⁻¹ or less.

5. A purified anti-human cytomegalovirus envelope glycoprotein B antibody, wherein the concentration of antibody required for 50% inhibition of human cytomegalovirus is 25 ug/ml or less.

6. The antibody of claim 5, wherein the antibody binds to a polypeptide consisting of SEQ ID NO: 2.

7. A purified anti-human cytomegalovirus envelope glycoprotein B antibody that binds to the same epitope as or competes for binding to a polypeptide having an amino acid sequence consisting of SEQ ID NO: 2 with an antibody selected from the group consisting of:
(a) an antibody having a heavy chain amino acid sequence consisting of SEQ ID NO: 3 and a light chain amino acid sequence consisting of SEQ ID NO: 8, 9, 10, 11, 12, 13, 14, 15, 16, or 17;
(b) an antibody having a heavy chain amino acid sequence consisting of SEQ ID NO: 4 and a light chain amino acid sequence consisting of SEQ ID NO: 16;
(c) an antibody having a heavy chain amino acid sequence consisting of SEQ ID NO: 5 and a light chain amino acid sequence consisting of SEQ ID NO: 12;
(d) an antibody having a heavy chain amino acid sequence consisting of SEQ ID NO: 6 and a light chain amino acid sequence consisting of SEQ ID NO: 18 or 19; and
(e) an antibody having a heavy chain amino acid sequence consisting of SEQ ID NO: 7 and a light chain amino acid sequence consisting of SEQ ID NO: 20.

8. The antibody of any one of claims 1-7, wherein the antibody is human.

9. A polynucleotide that encodes a heavy or light chain of the antibody of any one of claims 1-7.

10. The polynucleotide of claim 9, wherein the polynucleotide comprises a sequence at least 80% identical to SEQ ID NO: 21, 30, 39, 48, 57, 66, 75, 84, 93, 102, 111, 120, 129, 138, 147, 156, 165, or 174.

11. A vector comprising the polynucleotide of claim 9.

12. An isolated cell comprising the polynucleotide of claim 9.

13. A method of detecting a human cytomegalovirus in a sample, the method comprising:
contacting a sample with the antibody of any one of claims 1-7; and
detecting binding of the antibody to the sample, thereby detecting a human cytomegalovirus in the sample.

14. A method of inhibiting human cytomegalovirus infection of a cell, the method comprising contacting the cell with the antibody of any one of claims 1-7.

15. The antibody of any one of claims 1-7 for treatment, prophylaxis, or diagnosis of a human cytomegalovirus infection.

16. A therapeutic, prophylactic, or diagnostic composition for human cytomegalovirus infection or for a human cytomegalovirus-related disease, comprising the antibody of any one of claims 1-7.

17. Use of the antibody of any one of claims 1-7 for treatment, prophylaxis, or diagnosis of a human cytomegalovirus infection.

18. Use of the antibody of any one of claims 1-7 in the manufacture of a medicament for treatment, prophylaxis, or diagnosis of a human cytomegalovirus infection.
